(19) United States
(12) Patent Application Publication
(10) Pub. No.: US 2021/0304896 A1
(43) Pub. Date: Sep. 30, 2021

(54) SYSTEMS AND METHODS FOR MEDICAL DIAGNOSIS

(71) Applicant: SHANGHAI UNITED IMAGING INTELLIGENCE CO., LTD., Shanghai (CN)

(72) Inventors: Yanbo CHEN, Shanghai (CN); Yaozong GAO, Shanghai (CN); Yanping YANG, Shanghai (CN)

(73) Assignee: SHANGHAI UNITED IMAGING INTELLIGENCE CO., LTD., Shanghai (CN)

(21) Appl. No.: 17/218,201

(22) Filed: Mar. 31, 2021

(30) Foreign Application Priority Data
Mar. 31, 2020 (CN) 202010240034.3
Mar. 31, 2020 (CN) 202010240210.3

(51) Int. Cl. G16H 50/20; G06T 7/11; G06K 9/46; G06K 9/62; G16H 30/40

(52) U.S. Cl. CPC G16H 50/20; G06T 7/11; G06K 9/4671; A61B 6/5217; G06K 9/6262; G16H 30/40; G06K 2209/051; G06K 9/6256

(57) ABSTRACT

A method is provided. The method may also include generating at least one first segmentation image and at least one second segmentation image based on the target image. Each of the at least one first segmentation image may indicate one of the at least one target region of the subject. Each of the at least one second segmentation image may indicate a lesion region of one of the at least one target region. The method may also include determining first feature information relating to the at least one lesion region and the at least one target region based on the at least one first segmentation image and the at least one second segmentation image. The method may further include generating a diagnosis result with respect to the subject based on the first feature information.

### CROSS-REFERENCE TO RELATED APPLICATION

This application claims priority to Chinese Patent Application No. 202010240034.3 and Chinese Patent Application No. 202010240210.3, both filed on Mar. 31, 2020, the contents of each of which are hereby incorporated by reference.

### TECHNICAL FIELD

The present disclosure generally relates to image processing, and more particularly, to methods and systems for medical diagnosis via image processing.

### BACKGROUND

An accurate detection and diagnosis of a disease (e.g., a lung disease) are vital for human health. With the development of medical imaging techniques, medical images are often acquired and serve as a basis of medical diagnosis. Thus, it is desirable to provide systems and methods for medical diagnosis via image processing.

### SUMMARY

According to an aspect of the present disclosure, a system may be provided. The system may include at least one storage device and at least one processor configured to communicate with the at least one storage device. The at least one storage device may include a set of instructions. When the at least one processor executes the set of instructions, the at least one processor may be directed to cause the system to perform one or more of the following operations. The system may obtain a target image of a subject including at least one target region. The system may also generate at least one first segmentation image and at least one second segmentation image based on the target image. Each of the at least one first segmentation image may indicate one of the at least one target region of the subject. Each of the at least one second segmentation image may indicate a lesion region of one of the at least one target region. The system may also determine first feature information relating to the at least one lesion region and the at least one target region based on the at least one first segmentation image and the at least one second segmentation image. The system may further generate a diagnosis result with respect to the subject based on the first feature information.

In some embodiments, the target image may be a medical image of the lungs of the subject, and the at least one target region includes at least one of the left lung, the right lung, a lung lobe, or a lung segment of the subject.

In some embodiments, the diagnosis result with respect to the subject may include a severity of illness of the subject or at least one target case. Each of the at least one target case may relate to a reference subject having a similar disease to the subject.

In some embodiments, to generate at least one first segmentation image and at least one second segmentation image based on the target image, the system may generate the at least one first segmentation image by processing the target image using a first segmentation model for segmenting the at least one target region. The system may further generate the at least one second segmentation image by processing the at least one first segmentation image and the target image using a second segmentation model for segmenting the at least one lesion region.

In some embodiments, to generate at least one first segmentation image and at least one second segmentation image based on the target image, the system may generate the at least one first segmentation image by processing the target image using a first segmentation model for segmenting the at least one target region. The system may also generate the at least one second segmentation image by processing the target image using a third segmentation model for segmenting the at least one lesion region.

In some embodiments, to determine first feature information relating to the at least one lesion region and the at least one target region based on the at least one first segmentation image and the at least one second segmentation image, the system may perform one or more of the following operations. For each of the at least one lesion region, the system may determine a lesion ratio of the lesion region to the target region corresponding to the lesion region based on the second segmentation image of the lesion region and the first segmentation image of the target region corresponding to the lesion region. The system may also determine a HU value distribution of the lesion region. The system may further determine the first feature information based on the lesion ratio and the HU value distribution of the lesion region.

In some embodiments, to generate a diagnosis result with respect to the subject based on the first feature information, the system may obtain second feature information of the subject. The second feature information may include clinical information of the subject. The system may further generate the diagnosis result with respect to the subject based on the first feature information and the second feature information.

In some embodiments, to generate the diagnosis result with respect to the subject based on the first feature information and the second feature information, the system may generate third feature information of the subject based on the first feature information and the second feature information. The system may further determine a severity of illness of the subject by processing the third feature information using a severity degree determination model.

In some embodiments, to generate the severity degree determination model, the system may obtain at least one training sample each of which includes sample feature information of a sample subject and a ground truth severity of illness of the sample subject. The sample feature information of the sample subject may include sample first feature information relating to at least one sample lesion region and at least one sample target region of the sample subject, and sample second feature information of the sample subject. The system may further generate the severity degree determination model by training a preliminary model using the at least one training sample.

In some embodiments, to generate a diagnosis result with respect to the subject based on the first feature information, the system may generate the diagnosis result with respect to the subject by processing the first feature information using a diagnosis result generation model.

In some embodiments, to determine first feature information relating to the at least one lesion region and the at least one target region based on the at least one first segmentation image and the at least one second segmentation image, the system may generate the first feature information by processing the at least one first segmentation image and the at least one second segmentation image using a feature extraction model. The feature extraction model and the diagnosis result generation model may be jointly trained using a machine learning algorithm.

In some embodiments, to generate a diagnosis result with respect to the subject based on the first feature information, the system may obtain a plurality of reference cases. Each of the plurality of reference cases may include reference feature information relating to at least one lesion region and at least one target region of a reference subject. The system may further select at least one target case based on the reference feature information of the plurality of reference cases and the first feature information from the plurality of reference cases. The reference subject of each of the at least one target case may have a similar disease to the subject.

In some embodiments, the reference feature information of each of the plurality of reference cases may be represented as a reference feature vector. To select at least one target case from the plurality of reference cases, the system may determine a feature vector representing the first feature information of the subject based on the first feature information. The system may further determine the at least one target case based on the plurality of reference feature vectors and the feature vector.

In some embodiments, to determine the at least one target case based on the plurality of reference feature vectors and the feature vector, the system may determine the at least one target case according to a Vector Indexing algorithm based on the plurality of reference feature vectors and the feature vector.

In some embodiments, to determine first feature information relating to the at least one lesion region and the at least one target region based on the at least one first segmentation image and the at least one second segmentation image, the system may determine initial first feature information based on the at least one first segmentation image and the at least one second segmentation image. The system may further generate the first feature information by preprocessing the initial first feature information. The preprocessing of the initial first feature information may include at least one of a normalization operation, a filtering operation, or a weighting operation.

According to another aspect of the present disclosure, a method may be provided. The method may include obtaining a target image of a subject including at least one target region. The method may also include generating at least one first segmentation image and at least one second segmentation image based on the target image. Each of the at least one first segmentation image may indicate one of the at least one target region of the subject. Each of the at least one second segmentation image may indicate a lesion region of one of the at least one target region. The method may also include determining first feature information relating to the at least one lesion region and the at least one target region based on the at least one first segmentation image and the at least one second segmentation image. The method may further include generating a diagnosis result with respect to the subject based on the first feature information.

According to yet another aspect of the present disclosure, a non-transitory computer readable medium may be provided. The non-transitory computer readable may include a set of instructions. When executed by at least one processor of a computing device, the set of instructions may cause the computing device to perform a method. The method may also include generating at least one first segmentation image and at least one second segmentation image based on the target image. Each of the at least one first segmentation image may indicate one of the at least one target region of the subject. Each of the at least one second segmentation image may indicate a lesion region of one of the at least one target region. The method may also include determining first feature information relating to the at least one lesion region and the at least one target region based on the at least one first segmentation image and the at least one second segmentation image. The method may further include generating a diagnosis result with respect to the subject based on the first feature information.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary imaging system according to some embodiments of the present disclosure;

FIG. 2 is a schematic diagram illustrating exemplary hardware and/or software components of a computing device according to some embodiments of the present disclosure;

FIG. 3 is a schematic diagram illustrating exemplary hardware and/or software components of a mobile device according to some embodiments of the present disclosure;

FIGS. 4A and 4B are block diagrams illustrating exemplary processing devices according to some embodiments of the present disclosure;

FIG. 5 is a flowchart illustrating an exemplary process for generating a diagnosis result with respect to a subject according to some embodiments of the present disclosure;

FIG. 6 is a flowchart illustrating an exemplary process for generating at least one first segmentation image and at least one second segmentation image according to some embodiments of the present disclosure;

FIG. 7 is a flowchart illustrating an exemplary process for generating at least one first segmentation image and at least one second segmentation image according to some embodiments of the present disclosure:

FIG. 8 is a flowchart illustrating an exemplary process for determining first feature information relating to at least one lesion region and at least one target region according to some embodiments of the present disclosure:

FIG. 9 is a flowchart illustrating an exemplary process for determining a severity degree of illness of a subject according to some embodiments of the present disclosure;

FIG. 10 is a flowchart illustrating an exemplary process for determining at least one target case according to some embodiments of the present disclosure;

FIGS. 11A and 11B are schematic diagrams illustrating exemplary training processes of a feature extraction model and a diagnosis result generation model according to some embodiments of the present disclosure:

FIG. 12 illustrates an exemplary target image of a patient according to some embodiments of the present disclosure;

FIG. 13 illustrates an exemplary first segmentation image according to some embodiments of the present disclosure;

FIG. 14 illustrates an exemplary first segmentation image according to some embodiments of the present disclosure;

FIG. 15 illustrates an exemplary first segmentation image according to some embodiments of the present disclosure;

FIG. 16 illustrates an exemplary second segmentation image according to some embodiments of the present disclosure;

FIG. 17 illustrates an exemplary display result of a target case according to some embodiments of the present disclosure;

FIG. 18A illustrates exemplary morphological feature information relating to the lungs of a patient according to some embodiments of the present disclosure;

FIG. 18B illustrates exemplary a HU value distribution relating to the lungs of a patient according to some embodiments of the present disclosure; and

FIG. 19 illustrates exemplary similarities between feature information of reference cases and a subject according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections, or assembly of different levels in ascending order. However, the terms may be displaced by another expression if they achieve the same purpose.

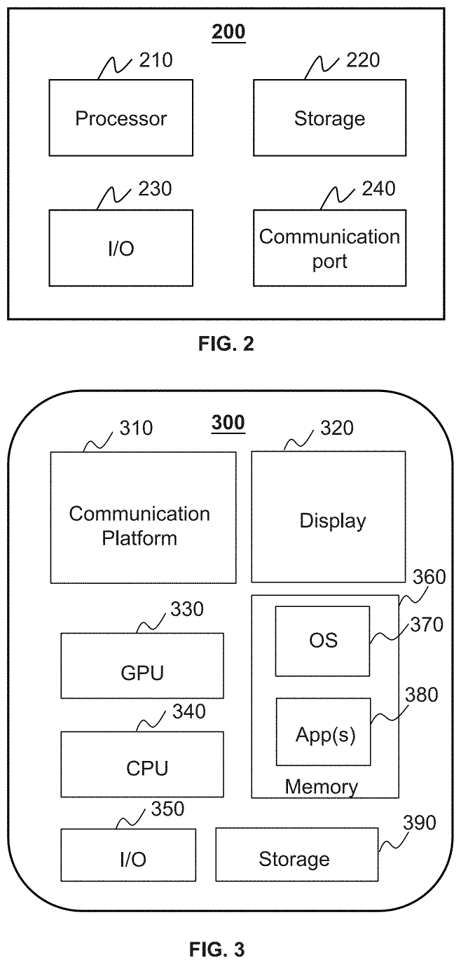

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., processor 210 as illustrated in FIG. 2) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The term “image” in the present disclosure is used to collectively refer to image data (e.g., scan data, projection data) and/or images of various forms, including a two-dimensional (2D) image, a three-dimensional (3D) image, a four-dimensional (4D), etc. The term “pixel” and “voxel” in the present disclosure are used interchangeably to refer to an element of an image. An anatomical structure shown in an image of a subject may correspond to an actual anatomical structure existing in or on the subject's body. The term “segmenting an anatomical structure” or “identifying an anatomical structure” in an image of a subject may refer to segmenting or identifying a portion in the image that corresponds to an actual anatomical structure existing in or on the subject's body.

These and other features, and features of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

Provided herein are systems and methods for non-invasive biomedical imaging, such as for disease diagnostic or research purposes. In some embodiments, the systems may include a single modality imaging system and/or a multi-modality imaging system. The single modality imaging system may include, for example, an ultrasound imaging system, an X-ray imaging system, an computed tomography (CT) system, a magnetic resonance imaging (MRI) system, an ultrasonography system, a positron emission tomography (PET) system, an optical coherence tomography (OCT) imaging system, an ultrasound (US) imaging system, an intravascular ultrasound (IVUS) imaging system, a near-infrared spectroscopy (NIRS) imaging system, a far-infrared (FIR) imaging system, or the like, or any combination thereof. The multi-modality imaging system may include, for example, an X-ray imaging-magnetic resonance imaging (X-ray-MRI) system, a positron emission tomography-X-ray imaging (PET-X-ray) system, a single-photon emission computed tomography-magnetic resonance imaging (SPECT-MRI) system, a positron emission tomography-computed tomography (PET-CT) system, a C-arm system, a digital subtraction angiography-magnetic resonance imaging (DSA-MRI) system, etc. It should be noted that the imaging system described below is merely provided for illustration purposes, and not intended to limit the scope of the present disclosure.

The term “imaging modality” or “modality” as used herein broadly refers to an imaging method or technology that gathers, generates, processes, and/or analyzes imaging information of a subject. The subject may include a biological subject and/or a non-biological subject. The biological subject may be a human being, an animal, a plant, or a portion thereof (e.g., a heart, a breast, etc.). In some embodiments, the subject may be a man-made composition of organic and/or inorganic matters that are with or without life.

Medical imaging techniques, such as a magnetic resonance imaging (MRI) technique, a computed tomography (CT) imaging technique, or the like, have been widely used for disease diagnosis and treatment. Sometimes, it is difficult to accurately detect and diagnose some diseases. For example, it is difficult to accurately detect and diagnose lung diseases (such as, a pneumonia, an emphysema, a pulmonary fibrosis) for some reasons (e.g., because lung diseases often have similar symptoms). Merely for illustration purposes, early signs of Corona Virus Disease 2019 (COVID-19) and an ordinary pneumonia are similar and it is difficult to accurately distinguish COVID-19 from an ordinary pneumonia. Thus, an accurate medical diagnosis is vital for human being and the society. As used herein, “medical diagnosis” (or referred to as disease diagnosis) may involve various operations for determining which disease that a subject has and/or analyzing the disease, such as determining a similar case for the subject, determining a severity of illness of the subject, etc.

An aspect of the present disclosure relates to systems and methods for generating a diagnosis result with respect to a subject. The systems may obtain a target image of the subject. The systems may also generate at least one first segmentation image and at least one second segmentation image based on the target image. The systems may further determine first feature information relating to at least one lesion region and at least one target region of the subject based on the at least one first segmentation image and the at least one second segmentation image. The systems may further generate the diagnosis result with respect to the subject based on the first feature information. The diagnosis result with respect to the subject may include a severity of illness of the subject and/or at least one target case. The at least one target case may relate to a reference subject having a similar disease to the subject.

In some embodiments, the systems may also obtain second feature information of the subject, such as clinical information, location information of the at least one lesion region, disease information of the subject, or the like, or any combination thereof. The diagnosis result may be generated based on disease signs obtained from medical images of the subject (including the at least one first segmentation image and the at least one second segmentation image) as well as the second feature information. Compared with a conventional approach for generating the diagnosis result based on only one or more second feature information (e.g., clinical information, historical disease information, and disease symptom information) of a subject, the systems and methods of the present disclosure may generate the diagnosis result based on the first feature information and the second feature information, which may improve the accuracy and reliability of the diagnosis result by using more information. In addition, compared with a conventional approach of generating the diagnosis result with a lot of user intervention, the systems and methods of the present disclosure may generate the diagnosis result with little or no user intervention, which is more reliable and robust, insusceptible to human error or subjectivity, and/or fully automated.

For illustration purposes, medical diagnosis systems and methods for lung diseases are described hereinafter. It should be noted that this is not intended to be limiting, and the medical diagnosis systems and methods may be applied to detect and/or analyze other diseases, such as liver diseases, etc.

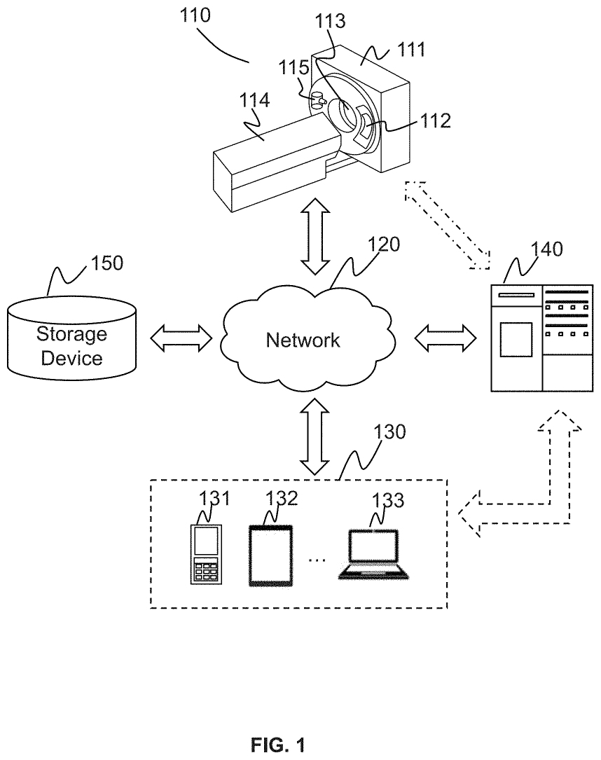

FIG. 1 is a schematic diagram illustrating an exemplary imaging system 100 according to some embodiments of the present disclosure. As shown, the imaging system 100 may include an imaging device 110, a network 120, one or more terminals 130, a processing device 140, and a storage device 150. In some embodiments, the imaging device 110, the terminal(s) 130, the processing device 140, and/or the storage device 150 may be connected to and/or communicate with each other via a wireless connection (e.g., the network 120), a wired connection, or a combination thereof. The connection between the components of the imaging system 100 may be variable. Merely by way of example, the imaging device 110 may be connected to the processing device 140 through the network 120, as illustrated in FIG. 1. As another example, the imaging device 110 may be connected to the processing device 140 directly or through the network 120. As a further example, the storage device 150 may be connected to the processing device 140 through the network 120 or directly.

The imaging device 110 may generate or provide image data related to a subject via scanning the subject. In some embodiments, the subject may include a biological subject and/or a non-biological subject. For example, the subject may include a specific portion of a body, such as a heart, a breast, or the like. In some embodiments, the imaging device 110 may include a single-modality scanner (e.g., an MRI device, a CT scanner, an X-ray imaging device) and/or multi-modality scanner (e.g., a PET-MRI scanner) as described elsewhere in this disclosure. In some embodiments, the image data relating to the subject may include projection data, one or more images of the subject, etc. The projection data may include raw data generated by the imaging device 110 by scanning the subject and/or data generated by a forward projection on an image of the subject.

In some embodiments, the imaging device 110 may include a gantry 111, a detector 112, a detecting region 113, a scanning table 114, and a radioactive scanning source 115. The gantry 111 may support the detector 112 and the radioactive scanning source 115. The subject may be placed on the scanning table 114 to be scanned. The radioactive scanning source 115 may emit radioactive rays to the subject. The radiation may include a particle ray, a photon ray, or the like, or a combination thereof. In some embodiments, the radiation may include a plurality of radiation particles (e.g., neutrons, protons, electron, p-mesons, heavy ions), a plurality of radiation photons (e.g., X-ray, a y-ray, ultraviolet, laser), or the like, or a combination thereof. The detector 112 may detect radiations and/or radiation events (e.g., gamma photons) emitted from the detecting region 113. In some embodiments, the detector 112 may include a plurality of detector units. The detector units may include a scintillation detector (e.g., a cesium iodide detector) or a gas detector. The detector unit may be a single-row detector or a multi-rows detector.

The network 120 may include any suitable network that can facilitate the exchange of information and/or data for the imaging system 100. In some embodiments, one or more components of the imaging system 100 (e.g., the imaging device 110, the processing device 140, the storage device 150, the terminal(s) 130) may communicate information and/or data with one or more other components of the imaging system 100 via the network 120. For example, the processing device 140 may obtain image data from the imaging device 110 via the network 120. As another example, the processing device 140 may obtain user instruction(s) from the terminal(s) 130 via the network 120.

The network 120 may be or include a public network (e.g., the Internet), a private network (e.g., a local region network (LAN)), a wired network, a wireless network (e.g., an 802.11 network, a Wi-Fi network), a frame relay network, a virtual private network (VPN), a satellite network, a telephone network, routers, hubs, switches, server computers, and/or any combination thereof. For example, the network 120 may include a cable network, a wireline network, a fiber-optic network, a telecommunications network, an intranet, a wireless local region network (WLAN), a metropolitan region network (MAN), a public telephone switched network (PSTN), a Bluetooth™ network, a ZigBee™ network, a near field communication (NFC) network, or the like, or any combination thereof. In some embodiments, the network 120 may include one or more network access points. For example, the network 120 may include wired and/or wireless network access points such as base stations and/or internet exchange points through which one or more components of the imaging system 100 may be connected to the network 120 to exchange data and/or information.

The terminal(s) 130 may be connected to and/or communicate with the imaging device 110, the processing device 140, and/or the storage device 150. For example, the terminal(s) 130 may receive a user instruction to generate a diagnosis result with respect to the subject. As another example, the terminal(s) 130 may display a diagnosis result with respect to the subject generated by the processing device 140. In some embodiments, the terminal(s) 130 may include a mobile device 131, a tablet computer 132, a laptop computer 133, or the like, or any combination thereof. For example, the mobile device 131 may include a mobile phone, a personal digital assistant (PDA), a gaming device, a navigation device, a point of sale (POS) device, a laptop, a tablet computer, a desktop, or the like, or any combination thereof. In some embodiments, the terminal(s) 130 may include an input device, an output device, etc. In some embodiments, the terminal(s) 130 may be part of the processing device 140.

The processing device 140 may process data and/or information obtained from the imaging device 110, the storage device 150, the terminal(s) 130, or other components of the imaging system 100. In some embodiments, the processing device 140 may be a single server or a server group. The server group may be centralized or distributed. For example, the processing device 140 may generate one or more trained models that can be used in medical diagnosis. As another example, the processing device 140 may apply the trained model(s) in medical diagnosis. In some embodiments, the trained model(s) may be generated by a processing device, while the application of the trained model(s) may be performed on a different processing device. In some embodiments, the trained model(s) may be generated by a processing device of a system different from the imaging system 100 or a server different from the processing device 140 on which the application of the trained model(s) is performed. For instance, the trained model(s) may be generated by a first system of a vendor who provides and/or maintains such trained model(s), while the medical diagnosis may be performed on a second system of a client of the vendor. In some embodiments, the application of the trained model(s) may be performed online in response to a request for medical diagnosis. In some embodiments, the trained model(s) may be generated offline.

In some embodiments, the trained model(s) may be generated and/or updated (or maintained) by, e.g., the manufacturer of the imaging device 110 or a vendor. For instance, the manufacturer or the vendor may load the trained model(s) into the imaging system 100 or a portion thereof (e.g., the processing device 140) before or during the installation of the imaging device 110 and/or the processing device 140, and maintain or update the trained model(s) from time to time (periodically or not). The maintenance or update may be achieved by installing a program stored on a storage device (e.g., a compact disc, a USB drive, etc.) or retrieved from an external source (e.g., a server maintained by the manufacturer or vendor) via the network 120. The program may include a new model or a portion of a model that substitutes or supplements a corresponding portion of the model.

In some embodiments, the processing device 140 may be local to or remote from the imaging system 100. For example, the processing device 140 may access information and/or data from the imaging device 110, the storage device 150, and/or the terminal(s) 130 via the network 120. As another example, the processing device 140 may be directly connected to the imaging device 110, the terminal(s) 130, and/or the storage device 150 to access information and/or data. In some embodiments, the processing device 140 may be implemented on a cloud platform. For example, the cloud platform may include a private cloud, a public cloud, a hybrid cloud, a community cloud, a distributed cloud, an inter-cloud, a multi-cloud, or the like, or a combination thereof. In some embodiments, the processing device 140 may be implemented by a computing device 200 having one or more components as described in connection with FIG. 2.

In some embodiments, the processing device 140 may include one or more processors (e.g., single-core processor(s) or multi-core processor(s)). Merely by way of example, the processing device 140 may include a central processing unit (CPU), an application-specific integrated circuit (ASIC), an application-specific instruction-set processor (ASIP), a graphics processing unit (GPU), a physics processing unit (PPU), a digital signal processor (DSP), a field-programmable gate array (FPGA), a programmable logic device (PLD), a controller, a microcontroller unit, a reduced instruction-set computer (RISC), a microprocessor, or the like, or any combination thereof.

The storage device 150 may store data, instructions, and/or any other information. In some embodiments, the storage device 150 may store data obtained from the processing device 140, the terminal(s) 130, and/or the imaging device 110. For example, the storage device 150 may store image data collected by the imaging device 110. As another example, the storage device 130 may store one or more images. As still example, the storage device 130 may store a diagnosis result with respect to the subject. In some embodiments, the storage device 150 may store data and/or instructions that the processing device 140 may execute or use to perform exemplary methods described in the present disclosure. For example, the storage device 150 may store data and/or instructions that the processing device 140 may execute or use for generating a diagnosis result.

In some embodiments, the storage device 150 may include a mass storage device, a removable storage device, a volatile read-and-write memory, a read-only memory (ROM), or the like, or any combination thereof. Exemplary mass storage devices may include a magnetic disk, an optical disk, a solid-state drive, etc. Exemplary removable storage devices may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 150 may be implemented on a cloud platform as described elsewhere in the disclosure.

In some embodiments, the storage device 150 may be connected to the network 120 to communicate with one or more other components of the imaging system 100 (e.g., the processing device 140, the terminal(s) 130). One or more components of the imaging system 100 may access the data or instructions stored in the storage device 150 via the network 120. In some embodiments, the storage device 150 may be part of the processing device 140.

It should be noted that the above description of the imaging system 100 is intended to be illustrative, and not to limit the scope of the present disclosure. Many alternatives, modifications, and variations will be apparent to those skilled in the art. The features, structures, methods, and other features of the exemplary embodiments described herein may be combined in various ways to obtain additional and/or alternative exemplary embodiments. For example, the imaging system 100 may include one or more additional components. Additionally or alternatively, one or more components of the imaging system 100 described above may be omitted. As another example, two or more components of the imaging system 100 may be integrated into a single component.

FIG. 2 is a schematic diagram illustrating exemplary hardware and/or software components of a computing device 200 according to some embodiments of the present disclosure. The computing device 200 may be used to implement any component of the imaging system 100 as described herein. For example, the processing device 140 and/or the terminal(s) 130 may be implemented on the computing device 200, respectively, via its hardware, software program, firmware, or a combination thereof. Although only one such computing device is shown, for convenience, the computer functions relating to the imaging system 100 as described herein may be implemented in a distributed fashion on a number of similar platforms, to distribute the processing load. As illustrated in FIG. 2, the computing device 200 may include a processor 210, a storage device 220, an input/output (I/O) 230, and a communication port 240.

The processor 210 may execute computer instructions (e.g., program code) and perform functions of the processing device 140 in accordance with techniques described herein. The computer instructions may include, for example, routines, programs, subjects, components, data structures, procedures, modules, and functions, which perform particular functions described herein. For example, the processor 210 may process image data obtained from the imaging device 110, the terminal(s) 130, the storage device 150, and/or any other component of the imaging system 100. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combinations thereof.

Merely for illustration, only one processor is described in the computing device 200. However, it should be noted that the computing device 200 in the present disclosure may also include multiple processors, thus operations and/or method operations that are performed by one processor as described in the present disclosure may also be jointly or separately performed by the multiple processors. For example, if in the present disclosure the processor of the computing device 200 executes both operation A and operation B, it should be understood that operation A and operation B may also be performed by two or more different processors jointly or separately in the computing device 200 (e.g., a first processor executes operation A and a second processor executes operation B, or the first and second processors jointly execute operations A and B).

The storage device 220 may store data/information obtained from the imaging device 110, the terminal(s) 130, the storage device 150, and/or any other component of the imaging system 100. In some embodiments, the storage device 220 may include a mass storage device, a removable storage device, a volatile read-and-write memory, a read-only memory (ROM), or the like, or any combination thereof. In some embodiments, the storage device 220 may store one or more programs and/or instructions to perform exemplary methods described in the present disclosure.

The I/O 230 may input and/or output signals, data, information, etc. In some embodiments, the I/O 230 may enable a user interaction with the processing device 140. In some embodiments, the I/O 230 may include an input device and an output device. The input device may include alphanumeric and other keys that may be input via a keyboard, a touch screen (for example, with haptics or tactile feedback), a speech input, an eye tracking input, a brain monitoring system, or any other comparable input mechanism. The input information received through the input device may be transmitted to another component (e.g., the processing device 140) via, for example, a bus, for further processing. Other types of the input device may include a cursor control device, such as a mouse, a trackball, or cursor direction keys, etc. The output device may include a display (e.g., a liquid crystal display (LCD), a light-emitting diode (LED)-based display, a flat panel display, a curved screen, a television device, a cathode ray tube (CRT), a touch screen), a speaker, a printer, or the like, or a combination thereof.

The communication port 240 may be connected to a network (e.g., the network 120) to facilitate data communications. The communication port 240 may establish connections between the processing device 140 and the imaging device 110, the terminal(s) 130, and/or the storage device 150. The connection may be a wired connection, a wireless connection, any other communication connection that can enable data transmission and/or reception, and/or any combination of these connections. The wired connection may include, for example, an electrical cable, an optical cable, a telephone wire, or the like, or any combination thereof. The wireless connection may include, for example, a Bluetooth™ link, a Wi-Fi™ link, a WiMax™ link, a WLAN link, a ZigBee™ link, a mobile network link (e.g., 3G, 4G, 5G), or the like, or a combination thereof. In some embodiments, the communication port 240 may be and/or include a standardized communication port, such as RS232, RS485, etc. In some embodiments, the communication port 240 may be a specially designed communication port. For example, the communication port 240 may be designed in accordance with the digital imaging and communications in medicine (DICOM) protocol.

FIG. 3 is a schematic diagram illustrating exemplary hardware and/or software components of a mobile device 300 according to some embodiments of the present disclosure. In some embodiments, one or more components (e.g., a terminal 130 and/or the processing device 140) of the imaging system 100 may be implemented on the mobile device 300.

As illustrated in FIG. 3, the mobile device 300 may include a communication platform 310, a display 320, a graphics processing unit (GPU) 330, a central processing unit (CPU) 340, an I/O 350, a memory 360, and a storage 390. In some embodiments, any other suitable component, including but not limited to a system bus or a controller (not shown), may also be included in the mobile device 300. In some embodiments, a mobile operating system 370 (e.g., iOS™, Android™, Windows Phone™) and one or more applications 380 may be loaded into the memory 360 from the storage 390 in order to be executed by the CPU 340. The applications 380 may include a browser or any other suitable mobile apps for receiving and rendering information relating to image processing or other information from the processing device 140. User interactions with the information stream may be achieved via the I/O 350 and provided to the processing device 140 and/or other components of the imaging system 100 via the network 120.

To implement various modules, units, and their functionalities described in the present disclosure, computer hardware platforms may be used as the hardware platform(s) for one or more of the elements described herein. A computer with user interface elements may be used to implement a personal computer (PC) or any other type of work station or terminal device. A computer may also act as a server if appropriately programmed.

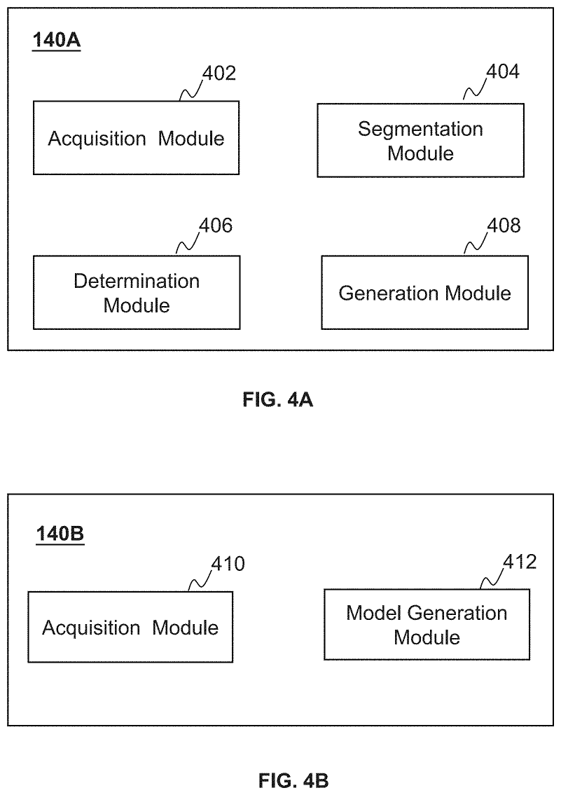

FIGS. 4A and 4B are block diagrams illustrating exemplary processing devices 140A and 140B according to some embodiments of the present disclosure. The processing devices 140A and 140B may be exemplary processing devices 140 as described in connection with FIG. 1. In some embodiments, the processing device 140A may be configured to generate a diagnosis result with respect to a subject. The processing device 140B may be configured to generate one or more machine learning models by model training.

In some embodiments, the processing device 140A may utilize the machine learning model(s) generated by the processing device 140B in the generation of the diagnosis result. In some embodiments, the processing devices 140A and 140B may be respectively implemented on a processing unit (e.g., a processor 210 illustrated in FIG. 2 or a CPU 340 as illustrated in FIG. 3). Merely by way of example, the processing devices 140A may be implemented on a CPU 340 of a terminal device, and the processing device 140B may be implemented on a computing device 200. Alternatively, the processing devices 140A and 140B may be implemented on a same computing device 200 or a same CPU 340. For example, the processing devices 140A and 140B may be implemented on a same computing device 200.

As shown in FIG. 4A, the processing device 140A may include an acquisition module 402, a segmentation module 404, a determination module 406, and a generation module 408.

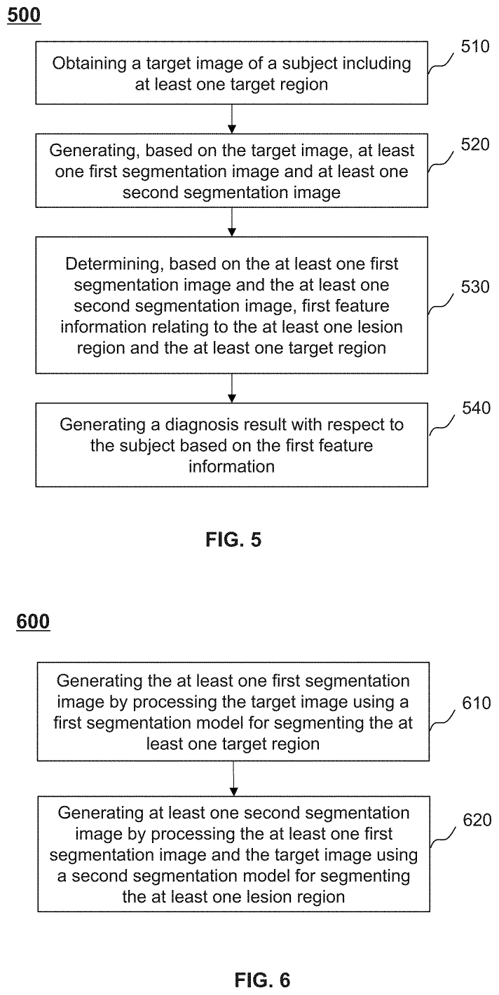

The acquisition module 402 may be configured to obtain information relating to the imaging system 100. For example, the acquisition module 402 may obtain a target image of a subject. As used herein, the subject may include a biological subject and/or a non-biological subject. For example, the subject may be a human being (e.g., an old person, a child, an adult, etc.), an animal, or a portion thereof. As another example, the subject may be a phantom that simulates a human. In some embodiments, the subject may be a patient or a portion thereof (e.g., the lungs). The subject may include at least one target region. In some embodiments, the subject may include a plurality of levels of target regions according to physiological anatomy. In some embodiments, the target image may include a 2D image (e.g., a slice image), a 3D image, a 4D image (e.g., a series of 3D images over time), and/or any related image data (e.g., scan data, projection data), or the like. In some embodiments, the target image may include a medical image generated by a biomedical imaging technique as described elsewhere in this disclosure. More descriptions regarding the obtaining of the target image of the subject may be found elsewhere in the present disclosure. See, e.g., operation 510 in FIG. 5, and relevant descriptions thereof.

The segmentation module 404 may be configured to generate at least one first segmentation image and at least one second segmentation image based on the target image. In some embodiments, each of the at least one first segmentation image may indicate one of the at least one target region of the subject. A second segmentation image may indicate a lesion region of one of the at least one target region. In some embodiments, a first segmentation image may indicate a plurality of target regions, and a second segmentation image may indicate lesion region(s) of the target regions. More descriptions regarding the generation of the at least one first segmentation image and the at least one second segmentation image may be found elsewhere in the present disclosure. See, e.g., operation 520 in FIG. 5, and relevant descriptions thereof.

The determination module 406 may be configured to make determinations. For example, the determination module 406 may be configured to determine first feature information relating to the at least one lesion region and/or the at least one target region based on the at least one first segmentation image and the at least one second segmentation image. For example, the first feature information may include distribution feature information, morphological feature information, density feature information, or the like, or any combination thereof. More descriptions regarding the determination of the first feature information may be found elsewhere in the present disclosure. See, e.g., operation 530 in FIG. 5, and relevant descriptions thereof.

The generation module 408 may be configured to generate the diagnosis result with respect to the subject based on the first feature information. The diagnosis result with respect to the subject may include information relate to the type, the feature, the symptom, or the like, of a disease of the subject and/or any other information that can facilitate the diagnosis of the diseases. For example, the diagnosis result with respect to the subject may include a severity of illness of the subject, at least one target case, disease progression of the subject, or the like, or any combination thereof. More descriptions regarding the generation of the diagnosis result may be found elsewhere in the present disclosure. See, e.g., operation 540 in FIG. 5, and relevant descriptions thereof.

As shown in FIG. 4B, the processing device 140B may include an acquisition module 410 and a model generation module 412.

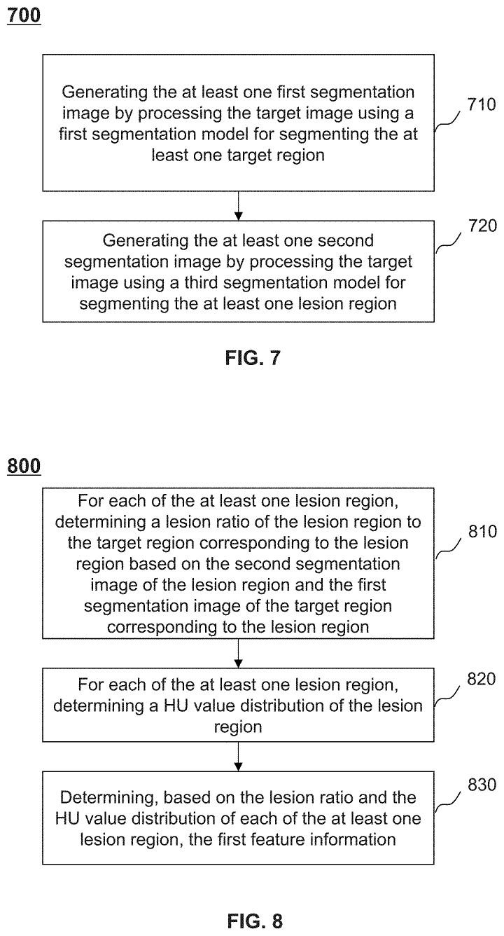

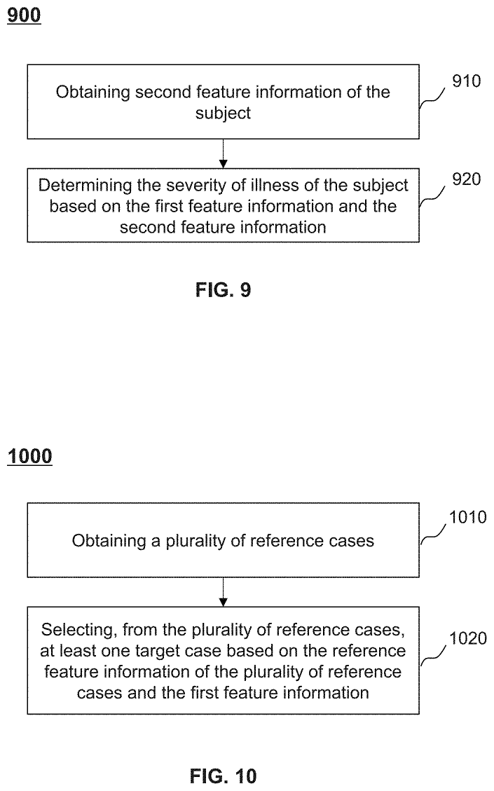

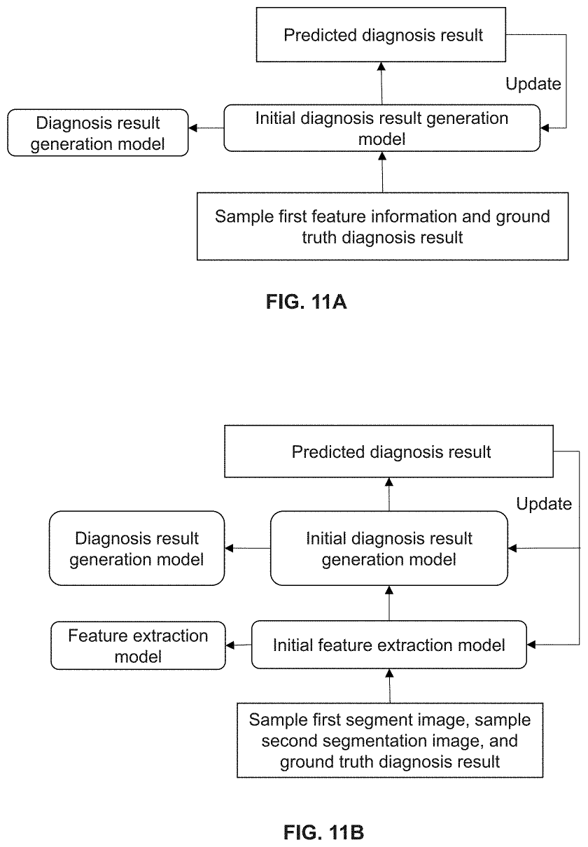

The acquisition module 410 may be configured to obtain one or more training samples and a corresponding preliminary model. More descriptions regarding the acquisition of the training samples and the corresponding preliminary model may be found elsewhere in the present disclosure. See, e.g., operations 610 and 620 in FIG. 6, operation 720 in FIG. 7, operation 920 in FIG. 9, FIGS. 11A and 11B, and relevant descriptions thereof.

The model generation module 410 may be configured to generate the one or more machine learning models by training a preliminary model using the more training samples. In some embodiments, the one or more machine learning models may be generated according to a machine learning algorithm. The machine learning algorithm may include but not be limited to an artificial neural network algorithm, a deep learning algorithm, a decision tree algorithm, an association rule algorithm, an inductive logic programming algorithm, a support vector machine algorithm, a clustering algorithm, a Bayesian network algorithm, a reinforcement learning algorithm, a representation learning algorithm, a similarity and metric learning algorithm, a sparse dictionary learning algorithm, a genetic algorithm, a rule-based machine learning algorithm, or the like, or any combination thereof. The machine learning algorithm used to generate the one or more machine learning models may be a supervised learning algorithm, a semi-supervised learning algorithm, an unsupervised learning algorithm, or the like. More descriptions regarding the generation of the one or more machine learning models may be found elsewhere in the present disclosure. See, e.g., operations 610 and 620 in FIG. 6, operation 720 in FIG. 7, operation 920 in FIG. 9, FIGS. 11A and 11B, and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the processing device 140A and/or the processing device 140B may share two or more of the modules, and any one of the modules may be divided into two or more units. For instance, the processing devices 140A and 140B may share a same acquisition module; that is, the acquisition module 402 and the acquisition module 410 are a same module. In some embodiments, the processing device 140A and/or the processing device 140B may include one or more additional modules, such as a storage module (not shown) for storing data. In some embodiments, the processing device 140A and the processing device 140B may be integrated into one processing device 140.

FIG. 5 is a flowchart illustrating an exemplary process for generating a diagnosis result with respect to a subject according to some embodiments of the present disclosure. In some embodiments, process 500 may be executed by the imaging system 100. For example, the process 500 may be implemented as a set of instructions (e.g., an application) stored in a storage device (e.g., the storage device 150, the storage device 220, and/or the storage 390). In some embodiments, the processing device 140A (e.g., the processor 210 of the computing device 200, the CPU 340 of the mobile device 300, and/or one or more modules illustrated in FIG. 4A) may execute the set of instructions and accordingly be directed to perform the process 500.

In 510, the processing device 140A (e.g., the acquisition module 402) may obtain a target image of the subject.

As used herein, the subject may include a biological subject and/or a non-biological subject. For example, the subject may be a human being (e.g., an old person, a child, an adult, etc.), an animal, or a portion thereof. As another example, the subject may be a phantom that simulates a human. In some embodiments, the subject may be a patient or a portion thereof (e.g., the lungs).

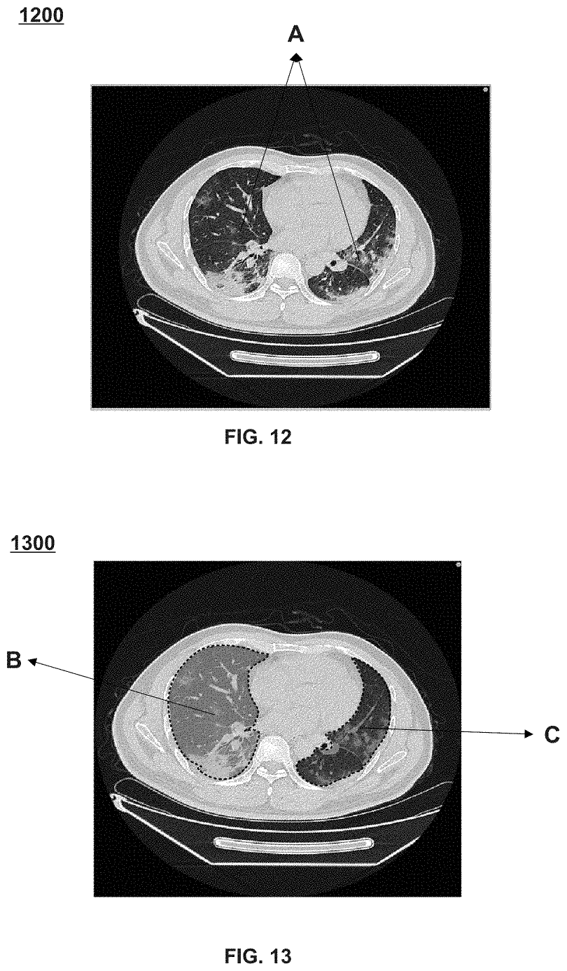

The subject may include at least one target region. A target region may include any region of interest of the subject. In some embodiments, a target region may be a region that encloses the lungs (or a portion thereof) of the subject. For example, the target region may include the whole lungs, the left lung, the right lung, one or more lung lobes, one or more lung segments of the subject, or the like, or any combination thereof. Merely for illustration purposes, FIG. 12 illustrates an exemplary target image 1200 of a patient according to some embodiments of the present disclosure. The target image 1200 is a CT image of the patient illustrating the lungs of the patient (denoted as regions A in FIG. 12).

In some embodiments, the subject may include a plurality of levels of target regions according to physiological anatomy. For example, the lungs of the subject may be divided into four levels. A first level may correspond to the whole lungs, a second level may correspond to the left lung or the right lung, a third level may correspond to the lung lobes, and a fourth level may correspond to the lung segments. The whole lungs may include the left lung and the right lung. Each of the left lung and the right lung may include multiple lung lobes. For example, the left lung may include 2 lung lobes (e.g., an upper lobe and a lower lobe), and the right lung may include 3 lung lobes (e.g., an upper lobe, a middle lobe, and a lower lobe). Each of the left lung and the right lung may include multiple lung segments. For example, the left lung may include 8 lung segments, and the right lung may include 10 lung segments. In some embodiments of the present disclosure, a target region of the subject refers to a certain level of target regions. For example, a target region of the subject may include a plurality of lung lobes of the subject. As another example, a target region of the subject may include a plurality of lung segments of the subject.

In some embodiments, the target image may include a 2D image (e.g., a slice image), a 3D image, a 4D image (e.g., a series of 3D images over time), and/or any related image data (e.g., scan data, projection data), or the like. In some embodiments, the target image may include a medical image generated by a biomedical imaging technique as described elsewhere in this disclosure. For example, the target image may include a DR image, an MR image, a PET image, a CT image, a PET-CT image, a PET-MR image, an ultrasound image, etc. In some embodiments, the target image may include a single image or a set of images of the subject. For example, the target image may include multiple medical images of the subject obtained with different imaging parameters (different scan sequences, different imaging modalities, different postures of the target image, etc.).

In some embodiments, the target image may be generated based on image data acquired using the imaging device 110 of the imaging system 100 or an external imaging device. For example, the imaging device 110, such as a CT device, an MRI device, an X-ray device, a PET device, or the like, may be directed to scan the subject or a portion of the subject (e.g., the chest of the subject). The processing device 140A may generate the target image based on image data acquired by the imaging device 110. In some embodiments, the target image may be previously generated and stored in a storage device (e.g., the storage device 150, the storage device 220, the storage 390, or an external source). The processing device 140A may retrieve the target image from the storage device. In some embodiments, the target image may be generated by scanning an image (e.g., a lung image) using a scanner.

In 520, the processing device 140A (e.g., the segmentation module 404) may generate, based on the target image, at least one first segmentation image and at least one second segmentation image.

In some embodiments, each of the at least one first segmentation image may indicate one of the at least one target region of the subject. For example, a first segmentation image may be an image obtained by segmenting the target image based on a target region corresponding to the first segmentation image.

A second segmentation image may indicate a lesion region of one of the at least one target region. For example, a second segmentation image may be obtained by processing the target image and/or one of the at least one first segmentation image. A lesion region may be a region of the subject that has damage (or potential damage) or an abnormal change, usually caused by disease or trauma. For example, a part of the left lung infected with a pneumonia may be a lesion region of the left lung. In some embodiments, a lesion region may include a plurality of sub-lesion regions distributed in the target region corresponding to the lesion region. In some embodiments, a segmentation image (e.g., a first segmentation image or a second segmentation image) may be a segmentation mask or an anatomy image in which a segmented region is labelled or marked out.

In some embodiments, the subject may include the plurality of levels of target regions according to physiological anatomy as aforementioned. The at least one first segmentation image may include a plurality of first segmentation images corresponding to the plurality of levels of target regions. In some embodiments, the at least one first segmentation image may include 26 first segmentation images each of which corresponds to one of the whole lungs, the left lung, the right lung, the 5 lung lobes, and the 18 lung segments. Similarly, the at least one second segmentation image may include multiple second segmentation images corresponding to the plurality of levels of target regions. For example, the at least one second segmentation image may include 26 second segmentation images each of which corresponds to one of the whole lungs, the left lung, the right lung, the 5 lung lobes, and the 18 lung segments. In some embodiments, the lungs of the subject may be divided into three levels including the second level, the third level, and the fourth level. In such cases, the at least one first segmentation image may include 25 images, and the at least one second segmentation image may also include 25 images.

In some embodiments, a first segmentation image may indicate a plurality of target regions, and a second segmentation image may indicate lesion region(s) of the target regions. For example, a first segmentation image may indicate a level of target regions. Similarly, a second segmentation image may indicate lesion region(s) of a level of target regions. For example, the at least one first segmentation image may include 4 first segmentation images each of which corresponds to one of the four levels of the lungs of the subject. The at least one second segmentation image may also include 4 second segmentation images each of which corresponds to one of the four levels of the lungs of the subject.

In some embodiments, a first segmentation image may be generated by segmenting one or more target regions from the target image manually by a user (e.g., a doctor, an imaging specialist, a technician) by, for example, drawing a bounding box on the target image displayed on a user interface. Alternatively, the target image may be segmented by the processing device 140A automatically according to an image analysis algorithm (e.g., an image segmentation algorithm). For example, the processing device 140A may perform image segmentation on the target image using an image segmentation algorithm. Exemplary image segmentation algorithm may include a thresholding segmentation algorithm, a compression-based algorithm, an edge detection algorithm, a machine learning-based segmentation algorithm, or the like, or any combination thereof. Alternatively, the at least one target region may be segmented by the processing device 140A semi-automatically based on an image analysis algorithm in combination with information provided by a user. Exemplary information provided by the user may include a parameter relating to the image analysis algorithm, a position parameter relating to a region to be segmented, an adjustment to, or rejection or confirmation of a preliminary segmentation result generated by the processing device 140A, etc.

In some embodiments, a plurality of first segmentation images corresponding to a plurality of levels of target regions may be generated directly by segmenting the target image.

Alternatively, the plurality of first segmentation images corresponding to the plurality of levels of target regions may be generated one by one based on the target image. Specifically, one or more first segmentation images corresponding to a certain level of target regions may be used to generate one or more first segmentation images corresponding to the next level of target regions. For example, one or more first segmentation images corresponding to one or more lung segments of a lung lobe may be generated by processing a first segmentation image corresponding to the lung lobe. By generating the first segmentation image(s) corresponding to the next level based on the first segmentation image(s) of the certain level instead of the original target image, the computation amount may be reduced, which may improve the segmentation efficiency and save computing resources. In addition, the segmentation accuracy may be improved. In some embodiments, the one or more first segmentation images corresponding to a certain level of target regions may be segmentation mask(s) corresponding to the certain level of target regions. The one or more first segmentation images corresponding the next level of target regions may need to be generated based on the one or more first segmentation images corresponding to the certain level of target regions and the target image.

A second segmentation image may be generated by segmenting a lesion region from the target image and/or a first segmentation image manually by a user, automatically by the processing device 140A, or semi-automatically. For example, the generation of the second segmentation image may be performed in a similar manner as that of the first segmentation image, and the descriptions thereof are not repeated here.

In some embodiments, the at least one first segmentation image may be generated by segmenting the at least one target region by processing the target image using a first segmentation model. The at least one second segmentation image may be generated by segmenting the at least one lesion region by processing the at least one first segmentation image and the target image using a second segmentation model, or by processing the target image using a third segmentation model. More descriptions for the generation of at least one first segmentation image and at least one second segmentation image using the first segmentation model, the second segmentation model, and the third segmentation model may be found elsewhere in the present disclosure (e.g., FIGS. 6 and 7 and the descriptions thereof).

According to some embodiments of the present disclosure, distribution information of the lung lobes and the lung segments in the lungs may be obtained according to the first segmentation images corresponding to the lung lobes and the lung segments, which may facilitate subsequent segmentation and positioning of the at least one lesion region. In such cases, lesion regions of the at least one target region may be observed and analyzed more clearly based on the locations of the lung lobes and the lung segments, thereby obtaining more accurate diagnosis results.

In 530, the processing device 140A (e.g., the determination module 406) may determine, based on the at least one first segmentation image and the at least one second segmentation image, first feature information relating to the at least one lesion region and/or the at least one target region.

For example, the first feature information may include distribution feature information, morphological feature information, density feature information, or the like, or any combination thereof.

The distribution feature information may indicate a distribution, such as a location distribution, a value distribution, etc., of a lesion region and/or a target region. Merely by way of example, the distribution feature information of a lesion region may indicate an image value distribution of the lesion region within an image value range (e.g., a pixel or voxel value range) of the lesion region. For example, if an image value range of the lesion region is [100, 200], the value distribution of the pixels or voxels of the lesion region within the image value range may be determined.

The morphological feature information may relate to the shape, the size, the volume, or the like, or any combination thereof, of a lesion region and/or a target region. Merely by way of example, the morphological feature information may include a lesion volume of a target region, a lesion volume of a lesion region of the target region, a lesion ratio of the target region, a lesion ratio of the lesion region of the target region, or the like, or any combination thereof. The lesion volume of a target region or a lesion region of the target region may be a volume of the lesion region in the target region. The lesion ratio of the target region or the lesion region may be a lesion ratio of a lesion region in the target region to the target region. Taking the lungs as an example, the morphological feature information relating to the lungs may include a lesion volume of the whole lungs, a lesion ratio of the whole lungs, a lesion volume of the left lung, a lesion volume of the right lung, a lesion ratio of the left lung, a lesion ratio of the right lung, a lesion volume of a lung lobe, a lesion ratio of a lung lobe, a lesion volume of a lung segment, a lesion ratio of a lung segment, or the like, or any combination thereof.

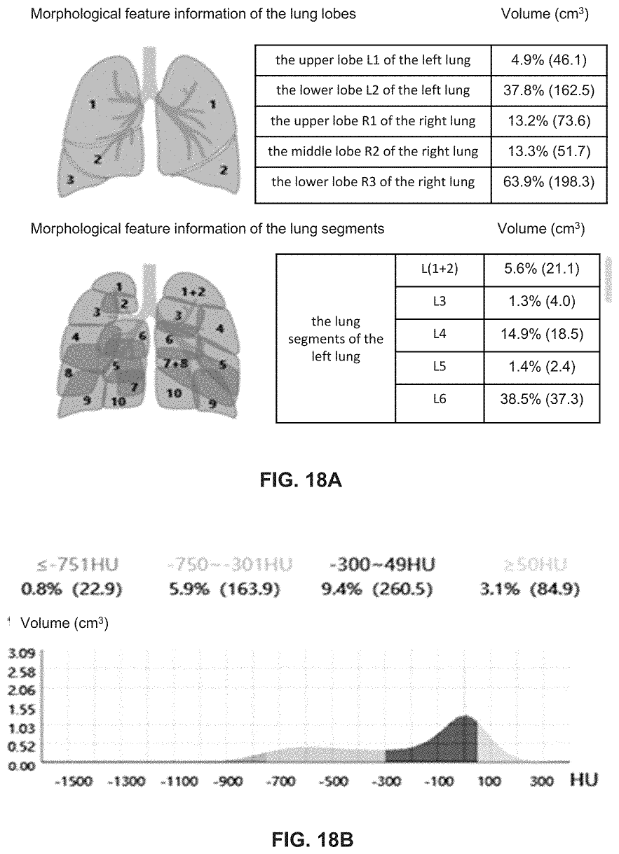

For illustration purposes, FIG. 18A illustrates exemplary morphological feature information relating to the lungs of a patient according to some embodiments of the present disclosure. For example, as shown in FIG. 18A, the morphological feature information of the upper lobe L1 of the left lung indicates that the lesion ratio of the upper lobe L1 of the left lung is 4.9%, and the lesion volume of the upper lobe L1 of the left lung is 46.1 cm³; the morphological feature information of the lung segment L(1+2) of the left lung indicates that the lesion ratio of the lung segment L(1+2) of the left lung is 5.6% and the lesion volume of the lung segment L(1+2) of the left lung is 21.1 cm³.

The density feature information may relate to the density of a lesion region and/or a target region. In some embodiments, the density feature information may include a Hounsfield Unit (HU) value distribution of a target region and/or a lesion region. A HU value may be generally used as a unit of a CT value, and indicate an X-ray absorption degree of a tissue (e.g., an X-ray absorption degree of the lungs), i.e., an X-ray attenuation coefficient corresponding to the tissue. After a CT image is obtained by scanning the subject with a CT scanning device, the HU value of each point may be obtained based on the CT image. Because that the HU value of a tissue may be associated with the density of the tissue, and the HU value distribution of a target region and/or a lesion region may be used as the density feature information of the target region and/or the lesion region.

Taking the lungs as an example, the density feature information may include a HU value distribution of the whole lungs, a HU value distribution of a lesion region of the whole lungs, a HU value distribution of the left lung, a HU value distribution of the right lung, a HU value distribution of a lesion region of the left lung, a HU value distribution of a lesion region of the right lung, a HU value distribution of a lung lobe, a HU value distribution of a lesion region of a lung lobe, a HU value distribution of a lung segment, a HU value distribution of a lesion region of a lung segment, or the like, or any combination thereof. In some embodiments, the HU value distribution of a target region or a lesion region may be represented by a graph (e.g., a histogram), a table, a curve, or the like, or any combination thereof.

Since HU value ranges corresponding to different tissues (e.g., different target regions) or components (e.g., a liver, a muscle, calcium, blood, or a plasma, etc.) are different, different HU value ranges may be used to represent different tissues. In some embodiments, a HU value range may be divided into a plurality of HU value intervals, and a HU value distribution of a target region or a lesion region in each HU value interval may be determined and used as the density feature information of the target region or the lesion region. For example, FIG. 18B illustrates exemplary a HU value distribution relating to the lungs of a patient according to some embodiments of the present disclosure. As shown in FIG. 18B, a HU value range [−1,500, 300] are divided into four HU value intervals including [−1500, −751], [−750, −301], [−300, 49], and [50, 300]. The four HU value intervals may represent different tissues or tissue components. A lesion volume and a lesion ratio of the lungs of the patient in each HU value interval may be determined. For example, as shown in FIG. 18B, a portion of the lungs within the HU value interval [−1500, −751] has a lesion volume of 22.9 cm³ and accounts for 0.8% of the total lesion volume of the lungs. More descriptions for the determination of the HU value distribution in a HU value interval may be found elsewhere in the present disclosure. See, e.g., operation 820 in FIG. 8 and relevant descriptions thereof.

In some embodiments, for each of the at least one lesion region, the processing device 140A may determine the first feature information based on the lesion volume, the lesion ratio, and the HU value distribution corresponding to the lesion region. More descriptions for the determination of the first feature information may be found elsewhere in the present disclosure. See, e.g., FIG. 8 and relevant descriptions thereof.

In some embodiments, the processing device 140A may determine initial first feature information based on the at least one first segmentation image and the at least one second segmentation image. In some embodiments, the initial first feature information may include the morphological feature information and the density feature information. The processing device 140A may further generate the first feature information by performing one or more preprocessing operations on the initial first feature information. The one or more preprocessing operations may include a normalization operation, a filtering operation, a weighting operation, or the like, or any combination thereof. More descriptions for the preprocessing operation(s) may be found elsewhere in the present disclosure. See, e.g., FIG. 9 and relevant descriptions thereof.

In some embodiments, the processing device 140A may determine the first feature information using a machine learning model. The machine learning model may be a trained feature extraction model. For example, the feature extraction model may be a deep learning network model, a support vector machine (SVM) model, a convolutional neural network (CNN) model, a recurrent neural network (RNN) model, etc. The processing device 140A may generate the first feature information by processing the at least one first segmentation image and the at least one second segmentation image using the trained feature extraction model. Detailed descriptions regarding the generation of the feature extraction model may be found elsewhere in the present disclosure. See, e.g., FIG. 11B and relevant descriptions thereof.

In 540, the processing device 140A (e.g., the generation module 408) may generate the diagnosis result with respect to the subject based on the first feature information.

The diagnosis result with respect to the subject may include information relate to the type, the feature, the symptom, or the like, of a disease of the subject and/or any other information that can facilitate the diagnosis of the diseases. For example, the diagnosis result with respect to the subject may include a severity of illness of the subject, at least one target case, disease progression of the subject, or the like, or any combination thereof.

The severity of illness of the subject may reflect a severity of a disease of the at least one lesion region of the subject. For example, if the at least one lesion region is a lung infection region and the disease of the at least one lesion region is a pneumonia, the severity of illness of the subject may reflect the severity of the pneumonia. In some embodiments, the severity of illness of the subject may be represented by a quantitative value of a quantitative index, wherein the quantitative index may be used to measure the severity of illness of a specific disease. For example, the quantitative value may be a value between 0-10, and different values may represent different severities of a disease (e.g., a pneumonia). Merely by way of example, 0 may represent that the severity of illness is the lowest, and 10 may represent that the severity of illness is the highest. A higher quantitative value may indicate higher severity of illness. As another example, the severity of illness of the subject may be represented as a risk degree of the disease, such as a low-risk degree, a moderate-risk degree, and a high-risk degree. In some embodiments, the quantitative value may also be represented in other ways, for example, using letters, etc., which is not intended to be limiting here.

A target case with respect to the subject used herein may relate to a reference subject having a similar disease to the subject. As used herein, two diseases may be deemed as similar diseases if they have the same or similar disease features (e.g., disease type, disease symptom, infected area, etc.). Detailed descriptions regarding the target case may be found elsewhere in the present disclosure. See, e.g., FIG. 10 and relevant descriptions thereof.

In some embodiments, the processing device 140A may determine the diagnosis result with respect to the subject based on a diagnosis result generation model (e.g., a machine learning model). For example, the processing device 140A may generate the diagnosis result with respect to the subject by processing the first feature information using the diagnosis result generation model. Detailed descriptions regarding the diagnosis result generation model may be found elsewhere in the present disclosure. See, e.g., FIGS. 11A and 11B and relevant descriptions thereof.

In some embodiments, the processing device 140A may obtain second feature information of the subject, such as clinical information of the subject, location information of the at least one lesion region, basic disease information relating to the disease of the subject, etc. The processing device 140A may further generate the diagnosis result with respect to the subject based on the first feature information and the second feature information. Detailed descriptions regarding the generation of the diagnosis result based on the first and second feature information may be found elsewhere in the present disclosure. See, e.g., FIG. 9 and relevant descriptions thereof.

According to some embodiments of the present disclosure, the processing device 140A may determine the diagnosis result with respect to the subject based on the first feature information relating to different lesion regions belonging to different target regions (e.g., lung parts with different levels), so that disease diagnosis of the subject may be performed at different levels, which may improve the accuracy of the diagnosis result.

FIG. 6 is a flowchart illustrating an exemplary process for generating at least one first segmentation image and at least one second segmentation image according to some embodiments of the present disclosure. In some embodiments, one or more operations of the process 600 may be performed to achieve at least part of operation 520 as described in connection with FIG. 5.

In 610, the processing device 140A (e.g., the segmentation module 404) may generate the at least one first segmentation image by processing the target image using a first segmentation model for segmenting the at least one target region.

As described in connection with operation 520, each of the at least one first segmentation image may indicate one of the at least one target region of the subject. For example, the at least one target region may include the whole lungs, the left lung, the right lung, one or more lung lobes, one or more lung segments of the subject, or the like, or any combination thereof.

In some embodiments, the subject may include multiple levels of target regions according to physiological anatomy. The processing device 140A may generate multiple first segmentation images corresponding to the multiple levels of target regions using the first segmentation model.

For example, the first segmentation model may include a plurality of segmentation units. Each segmentation unit of the first segmentation model may correspond to one of the levels of target regions and be used to generate the first segmentation image(s) of the corresponding level. For example, the first segmentation model may include a first segmentation unit, a second segmentation unit, a third segmentation unit, and a fourth segmentation unit, or any combination thereof. The first segmentation unit may be configured to segment the whole lungs of the subject from the target image. The second segmentation unit may be configured to segment the left lung and/or the right lung of the subject from the target image. The third segmentation unit may be configured to segment one or more lung lobes (e.g., an upper lobe of the left lung, a lower lobe of the left lung, an upper lobe of the right lung, a middle lobe of the right lung, the lower lobe of the right lung, etc., or any combination thereof) from the target image. The fourth segmentation unit may be configured to segment one or more lung segments (e.g., 8 lung segments of the left lung, 10 lung segments of the right lung) from the target image. In some embodiments, a segmentation unit of the first segmentation model may be a trained machine learning model. In some embodiments, the plurality of segmentation units may be stored as separate models and used to generate the first segmentation images. In other words, a plurality of first segmentation models corresponding to different levels of target regions may be acquired and used to generate the first segmentation images corresponding to multiple levels of target regions.

In some embodiments, the first segmentation model may be a shallow learning model or a deep learning model. The shallow learning model may include a Naive Bayes model, a decision tree model, a random forest model, an SVM model, etc. The deep learning model may include an artificial neural network model, such as a deep neural network (DNN) model, a CNN model, an RNN model, a feature pyramid network (FPN) model, etc.

In some embodiments, the first segmentation model may be a cascaded machine learning model including a plurality of segmentation models that are sequentially connected. The segmentation models may be configured to generate multiple first segmentation images corresponding to multiple levels of target regions one by one based on the target image. Each of the segmentation models may correspond to one of the multiple levels of target regions, and be configured to generate one or more first segmentation images corresponding to a certain level of the target region(s). The target image and an output of a specific segmentation model may be designated as an input of a next segmentation model connected with the specific segmentation model. Alternatively, an output of a specific segmentation model may be designated as an input of a next segmentation model connected with the specific segmentation model. For example, the first segmentation model may include a first-level segmentation model, a second-level segmentation model, a third-level segmentation model, and a fourth-level segmentation model arranged in sequence. The first-level segmentation model, the second-level segmentation model, the third-level segmentation model, and the fourth-level segmentation model may be configured to segment the whole lungs, the left lung and the right, the lung lobes, the lung segments, respectively. An input of the first-level segmentation model may include the target image and an output of the first-level segmentation model may include a first segmentation image A corresponding to the whole lungs. An input of the second-level segmentation model may include the first segmentation image A and an output of the second-level segmentation model may include a first segmentation image B corresponding to the left lung and a first segmentation image C corresponding to the right lung (or a single first segmentation image D corresponding to both the left and right lungs). An input of the third-level segmentation model may include one or more of the first segmentation images B, C, and D and an output of the third-level segmentation model may include one or more first segmentation images E each corresponding to one or more lung lobes. An input of the fourth-level segmentation model may include one or more of the first segmentation image(s) E and an output of the fourth-level segmentation model may include one or more first segmentation images F each corresponding to one or more lung segments. In some embodiments, the first segmentation images A, B, C, D, E may be segmentation masks, and the input of each of the second-level segmentation model, the third-level segmentation model, and the fourth-level segmentation model may further include the target image.

In some embodiments, the processing device 140A may obtain the first segmentation model from one or more components of the imaging system 100 (e.g., the storage device 150, the terminals(s) 130) or an external source via a network (e.g., the network 120). For example, the first segmentation model may be previously trained by a computing device (e.g., the processing device 140B), and stored in a storage device (e.g., the storage device 150, the storage device 220, and/or the storage 390) of the imaging system 100. The processing device 140A may access the storage device and retrieve the first segmentation model. In some embodiments, the first segmentation model may be generated according to a machine learning algorithm as described elsewhere in this disclosure (e.g., FIG. 4B and the relevant descriptions).

For example, the first segmentation model may be trained according to a supervised learning algorithm by the processing device 140B or another computing device (e.g., a computing device of a vendor of the first segmentation model). The processing device 140B may obtain one or more first training samples and a first preliminary model. Each first training sample may include a first sample image of a sample subject and an annotation regarding at least one sample target region in the first sample image. Merely by way of example, the at least one sample target region of the first sample image may include the whole lungs, the left lung, the right lung, the one or more lung lobes, or the one or more lung segments of the sample subject, and the annotation regarding the at least one sample target region may be provided or confirmed by a user as a ground truth segmentation result.

The training of the first preliminary model may include one or more first iterations to iteratively update model parameters of the first preliminary model based on the first training sample(s) until a first termination condition is satisfied in a certain iteration. Exemplary first termination conditions may include that the value of a first loss function obtained in the certain iteration is less than a threshold, that a certain count of iterations has been performed, that the first loss function converges such that the difference of the values of the first loss function obtained in a previous iteration and the current iteration is within a threshold value, etc. The first loss function may be used to measure a discrepancy between a segmentation result predicted by the first preliminary model in an iteration and the ground truth segmentation result. Exemplary first loss functions may include a focal loss function, a log loss function, a cross-entropy loss, a Dice ratio, or the like. If the first termination condition is not satisfied in the current iteration, the processing device 140B may further update the first preliminary model to be used in a next iteration according to, for example, a backpropagation algorithm. If the first termination condition is satisfied in the current iteration, the processing device 140B may designate the first preliminary model in the current iteration as the first segmentation model.

In some embodiments, as aforementioned, the first segmentation model may include a plurality of segmentation units or a plurality of segmentation models corresponding to a plurality of levels of target regions. In such cases, the first preliminary model may include a plurality of preliminary sub-models. The preliminary sub-models may be trained separately or jointly. For example, each first sample image may include annotations regarding different lung parts (e.g., the left and right lungs, the lung lobes, and the lung segments) of a sample subject, and the first sample image(s) may be used to train the preliminary sub-models simultaneously to generate the first, second, third, and fourth segmentation units of the first segmentation model. As another example, a set of first sample images having annotations regarding the upper lobe of the left lung may be used to generate a third segmentation unit for segmenting the upper lobe of the left lung. In some embodiments, the segmentation units or the segmentation models of the first segmentation model may be trained jointly (or in parallel), which improves the training efficiency of the first segmentation model.

In some embodiments, the processing device 140A may generate information related to the at least one target region (e.g., boundary information and/or location information of the at least one target region) based on the target image using the first segmentation model. The processing device 140A may further generate the at least one first segmentation image based on the information related to the at least one target region. Alternatively, the processing device 140A may directly generate the at least one first segmentation image by processing the target image using the first segmentation model.

In 620, the processing device 140A (e.g., the segmentation module 404) may generate at least one second segmentation image by processing the at least one first segmentation image and the target image using a second segmentation model for segmenting the at least one lesion region.

In some embodiments, if the at least one first segmentation image is anatomy image(s) in which a segmented region is labelled or marked out, the at least one second segmentation image may be generated by processing the at least one first segmentation image using the second segmentation model. If the at least one first segmentation image is segmentation mask(s), the at least one second segmentation image may be generated by processing the at least one first segmentation image in combination with the target image using the second segmentation model. For illustration purposes, the following descriptions describe examples in which the at least one second segmentation image is generated by processing the at least one first segmentation image and the target image.

A second segmentation image may indicate a lesion region of one of the at least one target region. A second segmentation image corresponding to a target region may be obtained by processing the first segmentation image corresponding to the target region and the target image using the second segmentation model (or a portion thereof). In some embodiments, the second segmentation model may be a trained machine learning model. The second segmentation model may segment (or detect) a lesion region of a target region by processing a first segmentation image corresponding to the target region and the target image. The second segmentation may be of any type of model (e.g., a machine learning model similar to the first segmentation model) as described elsewhere in this disclosure (e.g., operation 610 in FIG. 6 and the relevant descriptions).

In some embodiments, the subject may include multiple levels of target regions. The second segmentation model may include a plurality of lesion segmentation units M1. Each lesion segmentation unit M1 of the second segmentation model may correspond to one of the multiple levels of target regions and be used to generate the second segmentation image(s) of the corresponding level. For example, the second segmentation model may include a first lesion segmentation unit M1, a second lesion segmentation unit M1, a third lesion segmentation unit M1, a fourth lesion segmentation unit M1, or any combination thereof. The first lesion segmentation unit M1 may be configured to segment a lesion region of the whole lungs of the subject by processing the first segmentation image corresponding to the whole lungs and the target image. The second lesion segmentation unit M1 may be configured to segment lesion regions of the left lung and/or the right lung of the subject by processing the first segmentation image(s) corresponding to the left lung and/or the right lung and the target image. The third lesion segmentation unit M1 may be configured to segment lesion region(s) of one or more lung lobes of the subject by processing the first segmentation image(s) corresponding to the lung lobe(s) and the target image. The fourth lesion segmentation unit M1 may be configured to segment lesion region(s) of one or more lung segments of the subject by processing the first segmentation image(s) corresponding to the lung segment(s) and the target image. In some embodiments, a lesion segmentation unit M1 may be a trained machine learning model. In some embodiments, the plurality of lesion segmentation units M1 may be stored as separate models and used to generate the second segmentation images. In other words, a plurality of second segmentation models corresponding to different levels of target regions may be acquired and used to generate the second segmentation images corresponding to multiple levels of target regions.

In some embodiments, the obtaining of the second segmentation model may be performed in a similar manner as that of the first segmentation model as described elsewhere in this disclosure, and the descriptions thereof are not repeated here. In some embodiments, the second segmentation model may be trained according to a supervised learning algorithm by the processing device 140B or another computing device (e.g., a computing device of a vendor of the second segmentation model). Merely by way of example, the processing device 140B may obtain one or more second training samples and a second preliminary model. Each second training sample may include a second sample image of a sample target region of a sample subject and an annotation regarding a sample lesion region of the sample target region in the second sample image. In some embodiments, the training of the second preliminary model may be performed in a similar manner as that of the first preliminary model as described elsewhere in this disclosure, and the descriptions thereof are not repeated here.

In some embodiments, the processing device 140A may generate information related to the at least one lesion region (e.g., boundary information of the at least one lesion region) based on the at least one first segmentation image and the target image using the second segmentation model. The processing device 140A may generate the at least one second segmentation image based on the information related to the at least one lesion region.

In the process 600, the processing device 140A may generate the at least one first segmentation image and the at least one second segmentation image using the first segmentation model and the second segmentation model, respectively, which may improve the accuracy and/or efficiency of the generation of the at least one first segmentation image and the at least one second segmentation image.

FIG. 7 is a flowchart illustrating an exemplary process for generating at least one first segmentation image and at least one second segmentation image according to some embodiments of the present disclosure. In some embodiments, one or more operations of the process 700 may be performed to achieve at least part of operation 520 as described in connection with FIG. 5.

In 710, the processing device 140A (e.g., the segmentation module 404) may generate the at least one first segmentation image by processing the target image using a first segmentation model for segmenting the at least one target region.

In some embodiments, the operation 710 may be performed in a similar manner as operation 610 of the process 600 as illustrated in FIG. 6, the descriptions thereof are not repeated here.

In 720, the processing device 140A (e.g., the segmentation module 404) may generate the at least one second segmentation image by processing the target image using a third segmentation model for segmenting the at least one lesion region.

A second segmentation image may indicate a lesion region of one of the at least one target region. In some embodiments, the third segmentation model may be a trained machine learning model. The third segmentation model may segment (or detect) lesion regions in the target image. The third segmentation model may be of any type of model (e.g., a machine learning model similar to the first segmentation model) as described elsewhere in this disclosure (e.g., operation 610 in FIG. 6 and the relevant descriptions).

In some embodiments, the subject may include multiple levels of target regions. The third segmentation model may include a plurality of lesion segmentation units M2. Each lesion segmentation unit M2 of the third segmentation model may correspond to one of the multiple levels of target regions and be used to generate the second segmentation image(s) of the corresponding level. For example, the third segmentation model may include a first lesion segmentation unit M2, a second lesion segmentation unit M2, a third lesion segmentation unit M2, a fourth lesion segmentation unit M2, or any combination thereof. The first lesion segmentation unit M2 may be configured to segment a lesion region of the whole lungs of the subject from the target image. The second lesion segmentation unit M2 may be configured to segment lesion regions of the left lung and/or the right lung of the subject from the target image. The third lesion segmentation unit M2 may be configured to segment lesion region(s) of one or more lung lobes of the subject from the target image. The fourth lesion segmentation unit M2 may be configured to segment lesion region(s) of one or more lung segments of the subject from the target image. In some embodiments, a lesion segmentation unit M2 may be a trained machine learning model. In some embodiments, the plurality of lesion segmentation units M2 may be stored as separate models and used to generate the second segmentation images. In other words, a plurality of third segmentation models corresponding to different levels of target regions may be acquired and used to generate the second segmentation images corresponding to multiple levels of target regions.

In some embodiments, the third segmentation model may be configured to segment lesion regions of multiple target regions from the target image jointly. The processing device 140A may generate a plurality of second segmentation images corresponding to the target regions based on a plurality of first segmentation images corresponding to the target regions the and a segmentation image generated by the third segmentation model. For example, the target image, or the target image and a first segmentation image corresponding to the whole lungs may be input into the third segmentation model. The third segmentation model may generate a segmentation image indicating a lesion region of the left lung, a lesion region of the right lung, a lesion region of an upper lobe of the left lung, etc. The processing device 140A may generate a second segmentation image corresponding to the left lung based on the segmentation image and the first segmentation image corresponding to the left lung (e.g., the location of the left lung as indicated by the first segmentation image). Additionally or alternatively, the processing device 140A may generate a second segmentation image corresponding to an upper lobe of the left lung based on the segmentation image and the first segmentation image corresponding to the upper lobe of the left lung.

In some embodiments, the obtaining of the third segmentation model may be performed in a similar manner as that of the first segmentation model as described elsewhere in this disclosure, and the descriptions thereof are not repeated here.

In some embodiments, the third segmentation model may be trained according to a supervised learning algorithm by the processing device 140B or another computing device (e.g., a computing device of a vendor of the third segmentation model). Merely by way of example, the processing device 140B may obtain one or more third training samples and a third preliminary model. Each third training sample may include a third sample image of a sample subject and an annotation regarding a lesion region of each of at least one sample target region in the third sample image. In some embodiments, the training of the third preliminary model may be performed in a similar manner as that of the first preliminary model as described elsewhere in this disclosure, and the descriptions thereof are not repeated here.

In some embodiments, the processing device 140A may generate the at least one second segmentation image according to symptoms of the disease of the subject.

Compared with a conventional image segmentation approach which involves a lot of human intervention, the processes 600 and 700 that utilize the segmentation model(s) may be implemented with reduced user intervention, which improves the accuracy and/or generation efficiency of the at least one first segmentation image and the at least one second segmentation image.

FIG. 8 is a flowchart illustrating an exemplary process for determining first feature information relating to at least one lesion region and at least one target region according to some embodiments of the present disclosure. In some embodiments, one or more operations of the process 800 may be performed to achieve at least part of operation 530 as described in connection with FIG. 5.

In 810, for each of the at least one lesion region, the processing device 140A (e.g., the determination module 406) may determine a lesion ratio of the lesion region to the target region corresponding to the lesion region based on the second segmentation image of the lesion region and the first segmentation image of the target region corresponding to the lesion region.

In some embodiments, a lesion ratio of a lesion region to the target region corresponding to the lesion region may be a ratio of the volume of the lesion region to the volume of the target region corresponding to the lesion region. A target region corresponding to a lesion region refers to a target region where the lesion region locates. In some embodiments, a volume of a physical region of the subject may be represented by a count of voxels of an image region corresponding to the physical region. The processing device 140A may obtain a first count of voxels of the lesion region in the second segmentation image of the lesion region, and a second count of voxels of the target region corresponding to the lesion region in the first segmentation image of the target region. The processing device 140A may determine the lesion ratio of the lesion region to the target region corresponding to the lesion region based on the first count and the second count. In some embodiments, the processing device 140A may determine the first count and the second count using a voxel statistical tool or a voxel statistical method.

In some embodiments, the at least one target region may include 25 parts of the lungs of the subject (e.g., the left lung, the right lung, the 5 lung lobes, and the 18 lung segments), the at least one first segmentation image may include 25 first segmentation images corresponding to the 25 parts, and the at least one second segmentation image includes the 25 second segmentation images corresponding to the 25 parts. For each part of the lungs of the subject, the processing device 140A may determine the lesion ratio of the lesion region in the part to the part. For example, the processing device 140A may determine the lesion ratio of the lesion region in the left lung to the left lung, the lesion ratio of the lesion region in the right lung to the right lung, etc., thereby obtaining 25 lesion ratios. The 25 lesion ratios may reflect an infection status of the 25 parts of the lungs and a spread status of the at least one lesion region (e.g., a pneumonia infection region), and can be used for the subsequent determination of the severity of illness of the subject.

In 820, for each of the at least one lesion region, the processing device 140A (e.g., the determination module 406) may determine a HU value distribution of the lesion region.

In some embodiments, the HU value distribution of a lesion region may include a HU value distribution of the lesion region in multiple HU value intervals. The HU value intervals may be determined according to an actual need. The lengths of different HU value intervals may be the same or different. In some embodiments, a length of each of the HU value intervals may be determined according to a HU value range of the at least one first segmentation image and/or the at least one second segmentation image. For example, if the HU value range of the at least one second segmentation image is [−1150, 350] and the HU value range is divided into 30 HU value intervals, the length of each HU value interval may be 50. The 30 HU value intervals may include [−1150, −1100], [−1100, −1050], . . . , and [300, 350].

In some embodiments, the HU value distribution of a lesion region in the HU value intervals may be represented by distribution probability values each of which corresponds to one of the HU value intervals. A distribution probability value corresponding to a HU value interval may indicate a probability that the HU values of a lesion region belong to the HU value interval. Merely by way of example, the processing device 140A may obtain the HU value intervals and the HU value of each point (e.g., pixel or voxel) of the lesion region. The processing device 140A may match the HU value of each point of the lesion region with each HU value interval to obtain the count of points of the lesion region in each HU value interval. The processing device 140A may further obtain the distribution probability value of the lesion region in each HU value interval by normalizing the count of points of the lesion region in each HU value interval.

Specifically, a second segmentation image of a target region corresponding to the lesion region is obtained, the processing device 140A may obtain the HU value of each point of the lesion region. The processing device 140A may match the HU value of each point of the lesion region with each HU value interval to obtain the count of points of the lesion region in each HU value interval. The processing device 140A may further normalize the count of points of the lesion region in each HU value interval. For example, for a HU value interval, the processing device 140A may determine the distribution probability value corresponding to the HU value interval by dividing the count of points of the lesion region in the HU value interval by the total count of points of the lesion region.

For example, it is assumed that there are 3 HU value intervals, the count of points of a lesion region in the first HU value interval is 3, the count of points of the lesion region in the second HU value interval is 12, and the count of points of the lesion region in the third HU value interval is 5. The distribution probability values corresponding to the three HU value intervals are equal to 15%, 60%, and 25%, which are determined by dividing 3, 12, and 5 by a sum of 3, 12, and 5, respectively. By dividing the HU value range into multiple HU value intervals, more detailed feature information regarding the at least one lesion region of the subject may be obtained, which may better reflect the real situation of the at least one target region and the at least one lesion region of the subject.

In 830, the processing device 140A (e.g., the determination module 406) may determine, based on the lesion ratio and the HU value distribution of each of the at least one lesion region, the first feature information.

For example, the processing device 140A may designate the lesion ratio and the HU value distribution of each of the at least one lesion region as the first feature information. In some embodiments, the first feature information may include other information as described elsewhere in this disclosure (e.g., FIG. 5 and the relevant descriptions).

According to some embodiments of the present disclosure, the processing device 140A may determine a lesion ratio and/or a HU value distribution for each lesion region. The processing device 140A may further determine the lesion ratio and the HU value distribution as the first feature information based on the at least one lesion region and the at least one target region, which can simplify the determination of the first feature information and intuitively reflect the real situation of the at least one lesion region.

FIG. 9 is a flowchart illustrating an exemplary process for determining a severity degree of illness of a subject according to some embodiments of the present disclosure. In some embodiments, one or more operations of the process 900 may be performed to achieve at least part of operation 540 as described in connection with FIG. 5.

In 910, the processing device 140A (e.g., the generation module 408) may obtain second feature information of the subject.

In some embodiments, the second feature information may include clinical information of the subject, location information of the at least one lesion region, disease information of the subject, or the like, or any combination thereof.

The clinical information may include a height, a weight, an age, disease signs (e.g., fever, cough, etc.), a physical examination indicator (e.g., a body temperature, a blood pressure, a CURB-65 score value, a CRB-65 score value, a Pneumonia Severity Index (PSI), etc.), etc., or any combination thereof, of the subject. The CURB-65 score value, the CRB-65 score value, and the PSI may be associated with lung diseases, wherein C represents a consciousness disorder, U represents a uric acid nitrogen, R represents a respiratory rate, B represents a blood pressure, and 65 represents an age of the subject. In some embodiments, clinical information (e.g., a body temperature, a blood pressure) may be acquired at multiple time points since it may change over time.

The location information of a lesion region may include coordinate information of one or more points (e.g., a central point) of the lesion region, a target region corresponding to the lesion region, a relative position of the lesion region and the target region corresponding to the lesion region, or the like, or any combination thereof. For example, the relative position of the lesion region and the target region corresponding to the lesion region may indicate whether the lesion region is in a central part of the target region, at the edge of the target region, or partially within the target region, etc. The location information of the lesion region may be manually determined by a user or automatically determined by the processing device 140A based on the second segmentation image and/or the first segmentation image of the target region corresponding to the lesion region. For example, the processing device 140A may determine boundary information of the lesion region and boundary information of the target region corresponding to the lesion region based on the second segmentation image and the first segmentation image of the target region corresponding to the lesion region. The processing device 140A may further determine the relative position of the lesion region and the corresponding target region based on the boundary information of the lesion region and the boundary information of the corresponding target region.

The disease information of the subject may include the type of a disease (e.g., a historical disease, a current disease, a basic disease) that the subject has (e.g., a pneumonia, a lung cancer, etc.), a severity of the disease (e.g., mild, moderate, severe, etc.), a course of the disease, a recovery status of the disease (e.g., whether the historical disease recurs), symptom information of the disease, or the like, or any combination thereof. For example, if the disease of the subject is a pneumonia, according to the course of the disease of the subject, the disease of the subject may be classified as a chronic pneumonia, a persistent pneumonia, an acute pneumonia, etc. The basic disease refers to a disease that may induce other diseases. For example, the basic disease relating to a lung disease may include a hypertension, a diabetes, a cardiovascular disease, a chronic obstructive pulmonary disease (COPD), a cancer, a chronic kidney disease, a hepatitis B, an immunodeficiency disease, or the like, or any combination thereof.

In some embodiments, the second feature information may be manually determined by a user. For example, the clinical information of the subject may be obtained by a clinician or a nurse by inquiring the subject or using a measurement apparatus. Additionally or alternatively, the second feature information may be acquired from a measurement apparatus. For example, a physical examination indicator of the subject may be acquired by a specific measurement apparatus. Additionally or alternatively, the second feature information may be obtained by the processing device 140A from a storage device (e.g., a local storage device or an external storage device) that stores the second feature information. For example, the processing device 140A may retrieve the disease information of the subject from a database (e.g., a case file storage system of a hospital). Additionally or alternatively, the second feature information may be determined by the processing device 140A. For example, the disease information of the subject may be determined by the processing device 140A by analyzing one or more medical images of the subject. As another example, the processing device 140A may determine one or more of the CURB-65 score value, the CRB-65 score value, and the PSI of the subject by analyzing other clinical information of the subject.

In some embodiments, the second feature information may include the CURB-65 score value, the CRB-65 score value, and the PSI of the subject. The scoring system of the PSI is complex but the PSI has a high specificity for determining whether the subject needs to be hospitalized. The scoring system of the CURB-65 score value and the scoring system of the CRB-65 score value are relatively simple and sensitive, and have lower specificity. According to some embodiments of the present disclosure, various first feature information and second feature information relating to the subject (e.g., including all of the CURB-65 score value, the CRB-65 score value, and the PSI) may be obtained or determined and used in the subsequent determination of the severity of illness of the subject, thereby reducing a complexity of the system and improving the specificity of the system (e.g., being applicable to different subjects).

In 920, the processing device 140A (e.g., the generation module 408) may determine the severity of illness of the subject based on the first feature information and the second feature information.

In some embodiments, the processing device 140A may determine a first severity based on the first feature information. For example, for each feature information of the first feature information, the processing device 140A may determine a first score value corresponding to the feature information according to a first scoring rule. The processing device 140A may obtain a first weight value corresponding to each feature information of the first feature information. The processing device 140A may further determine the first severity based on the first score value and the first weight value corresponding to each feature information of the first feature information. In some embodiments, for different target regions and different types of disease, the first scoring rule and the first weight value corresponding to the first feature information may be different.

For example, the first feature information may include a lesion ratio and a HU value distribution corresponding to each of the left lung and the right lung of the subject. According to the first scoring rule, the processing device 140A may determine a first scoring value A and a first weight value a % corresponding to the lesion ratio of the left lung, a first scoring value B and a first weight value b % corresponding to the lesion ratio of the right lung, a first scoring value C and a first weight value c % corresponding to the HU value distribution of the left lung, and a first scoring value D and a first weight value d % corresponding to the HU value distribution of the right lung. The processing device 140A may determine the first severity by performing a weighted summation operation based on the first score values A, B, C, and D, and the first weight values a %, b %, c %, d %.

In some embodiments, the processing device 140A may determine a second severity based on the second feature information. In some embodiments, the determination of the second severity may be performed in a similar manner as the determination of the first severity level. For example, for each feature information of the second feature information, the processing device 140A may determine a second score value corresponding to the feature information according to a second scoring rule. The processing device 140A may obtain a second weight value corresponding to each feature information. The processing device 140A may further determine the second severity based on the second score value and the second weight value corresponding to each feature information in the second feature information.

In some embodiments, the processing device 140A may designate one of the first severity and the second severity as the severity of illness of the subject. Merely by way of example, the processing device 140A may determine a confidence level of the first severity and a confidence level of the second severity based on, for example, a source of the first and second feature information. The processing device 140A may select the one having a higher confidence level among the first severity and the second severity as the severity of illness of the subject. Alternatively, the processing device 140A may obtain a third weight value corresponding to the first severity and a fourth weight value corresponding to the second severity. The processing device 140A may determine the severity of illness of the subject by performing a weighted summation operation based on the first severity, the second severity, the third weight value, and the fourth weight value.

In some embodiments, the processing device 140A may determine the first severity and/or the second severity of a target region of the subject based on the first feature information and the second feature information relating to the target region. In some embodiments, the processing device 140A may determine the first severity and/or the second severity of each of a plurality of target regions of the subject, and determine the severity of illness of the subject based on the first severities and/or the second severities.

The first weight value, the second weight value, the third weight value, and the fourth weight value may be set manually by a user (e.g., an engineer) according to an experience value or a default setting of the imaging system 100, or determined by the processing device 140A according to an actual need. In some embodiments, the processing device 140A may determine an influence of each feature information on the severity of illness. For example, the processing device 140A may determine the influence of each feature information on the severity of illness by analyzing the feature information and the severity of illness of multiple similar cases. The processing device 140A may determine the weight value corresponding to each feature information based on the influence of each feature information on the severity of illness. For example, a higher weight value may be assigned to a specific type of feature information if the feature information has a high influence on the severity of illness.

In some embodiments, the processing device 140A may obtain a first severity degree determination model and a second severity degree determination model. The first feature information may be input into the first severity degree determination model, and the first severity degree determination model may output the first severity or information relating to the first severity. The second feature information may be input into the second severity determination mode, and the second severity degree determination model may output the second severity or information relating to the second severity. The processing device 140A may further determine the severity of illness of the subject based on the first severity and the second severity.

In some embodiments, the processing device 140A may obtain a third severity degree determination model. The third severity degree determination model may be configured to determine the severity of illness of the subject based on both the first feature information and the second feature information. Merely by way of example, the first feature information and the second feature information of the subject may be input to the third severity degree determination model, and the third severity degree determination model may output the severity of illness of the subject or information relating to the severity of illness of the subject.

In some embodiments, the processing device 140A may generate third feature information of the subject based on the first feature information and the second feature information. Merely by way of example, the processing device 140A may generate the third feature information of the subject by combining the first feature information and the second feature information. For example, the processing device 140A may generate first concatenated feature information by concatenating the first feature information relating to the at least one lesion region and the at least one target region. The processing device 140A may generate the third feature information by concatenating the first concatenated feature information and the second feature information. For example, it is assumed that the at least one lesion region includes two lesion regions, the first feature information relating to the two lesion regions are respectively represented as (x1, y1) and (x2, y2), and the second feature information is represented as (x3, y3, z1). The processing device 140A may concatenate the first feature information and the second feature information into (x1, y1, x2, y2, x3, y3, z1) (i.e., the third feature information). The concatenation sequence of the first and second feature information may be determined according to an actual need. The third feature information may be input into the third severity degree determination model, and the third severity degree determination model may output the severity of illness of the subject or information relating to the severity of illness.

In some embodiments, the processing device 140A may perform a selection operation on each feature information of the first feature information, the second feature information, or the third feature information to obtain selected feature information. The count of the selected feature information is not more than the count of the original feature information before selection. In some embodiments, the selection operation may be performed according to a reliability of each feature information, an influence of each feature information on the severity of illness, etc. The reliability of feature information may be determined based on an acquisition manner, the processing manner of the feature information, or the like, or any combination thereof. The influence of feature information on the severity of illness may be determined in a manner as aforementioned. In some embodiments, the processing device 140A may perform the selection operation according to a feature selection algorithm. Exemplary feature selection algorithm may include a low-variance feature selection algorithm, a least absolute shrinkage and selection operator (LASSO) algorithm, a univariate feature selection algorithm, a multivariate feature selection algorithm, a correlation coefficient algorithm, a chi-square test algorithm, a mutual information algorithm, or the like, or any combination thereof.

In some embodiments, the processing device 140A may perform the selection operation by performing a dimensionality reduction operation (e.g., using a principal component analysis algorithm). In some embodiments, the processing device 140A may perform a multi-level selection operation. In some embodiments, the selected feature information may have a higher precision and a better accuracy than the original feature information. The processing device 140A may determine the severity of illness of the subject based on the selected feature information, thereby generating a severity of illness of the subject with an improved accuracy and reliability. In addition, the severity of illness of the subject may be determined based on less amount of feature information, thereby improving the efficiency of the determination of the severity of illness by reducing the processing time and/or the needed processing resources.

In some embodiments, the processing device 140A may determine a final severity of illness based on the severities of illness determined in different manners and confidence coefficients corresponding to the severities of illness. The confidence coefficients may be set manually by a user (e.g., an engineer), according to an experience value, or according to a default setting of the imaging system 100, or determined by the processing device 140A. For example, the processing device 140A may obtain a first confidence coefficient and a second confidence coefficient. The first confidence coefficient may correspond to the severity of illness (also referred to as a third severity) determined by performing a weighted summation operation on the first severity and the second severity. The second confidence coefficient may correspond to the severity of illness (also referred to as a fourth severity) determined using the third severity degree determination model. The processing device 140A may further determine the final severity of illness by performing a weighted summation operation based on the third severity, the first confidence coefficient, the fourth severity, and the second confidence coefficient.

In some embodiments, one or more key factors influencing the severity of illness may be determined. Merely by way of example, the processing device 140A may determine a contribution score of the age of the subject to the severity of illness based on the second score value and the second weight value of the age of the subject, and the fourth weight value corresponding to the second severity (e.g., by multiplying the second score value, the second weight value, and the fourth weight value). The processing device 140A may designate one or more features with the largest N contribution score(s) as the key factor(s) influencing the severity of illness. As another example, the one or more key factors influencing the severity of illness may be determined based on model parameters of the third severity degree determination model (e.g., weights of different feature information). In this way, the severity of illness of the subject and the key factor(s) influencing the severity of illness may be determined at the same time, which improves the efficiency and/or accuracy of disease diagnosis, helps doctors make follow-up treatment plans faster, and enables patients to receive timely treatment.

In some embodiments, when the second feature information may include location information of the at least one lesion region, the processing device 140A may generate a risk prompt message according to the location information of the at least one lesion region. The risk prompt message may include information indicating one or more organs or tissues that are adjacent to the at least one lesion region and at risk of infection. In some embodiments, the processing device 140A may determine a risk level of an adjacent organ or tissue at risk of infection based on the determined severity of illness. The processing device 140A may prompt the user by sending the risk prompt message to a user terminal, and the user may make a treatment plan to reduce damages to the adjacent organ or tissue of the subject.

In some embodiments, the processing device 140A may obtain a severity degree determination model (e.g., the first, second, and third severity degree determination models) from one or more components of the imaging system 100 (e.g., the storage device 150, the terminals(s) 130) or an external source via a network (e.g., the network 120). For example, the severity degree determination model may be previously trained by a computing device (e.g., the processing device 140B), and stored in a storage device (e.g., the storage device 150, the storage device 220, and/or the storage 390) of the imaging system 100. The processing device 140A may access the storage device and retrieve the severity degree determination model. In some embodiments, the severity degree determination model may be generated according to a machine learning algorithm as described elsewhere in this disclosure (e.g., FIG. 4B and the relevant descriptions).

For illustration purposes, the generation of the third severity degree determination model is described hereinafter. The third severity degree determination model may be trained according to a supervised learning algorithm by the processing device 140B or another computing device (e.g., a computing device of a vendor of the third severity degree determination model). The processing device 140B may obtain at least one fourth training sample. Each fourth training sample may include sample feature information of a sample subject and a ground truth severity of illness of the sample subject. The sample feature information of the sample subject may include sample first feature information relating to at least one sample lesion region and at least one sample target region of the sample subject, and sample second feature information of the sample subject.

In some embodiments, for a fourth training sample, the processing device 140B may obtain a sample image of the sample subject of the fourth training sample. The sample images of different fourth training samples may be of a same type as or different types. Two images may be deemed as being of a same type if they are acquired using a same imaging modality. Acquisition times of the at least one sample image may be the same or different. The sample images of different fourth training samples may relate to a same sample subject or different sample subjects. Taking a pneumonia as an example, the at least one fourth training sample may include sample chest images of sample subjects with pneumonias of different severity, and/or a sample chest image of a normal sample subject without a pneumonia.

The processing device 140B may then determine the sample first feature information of the fourth training sample by performing operations 520 and 530 on the sample image. The processing device 140B may also obtain the sample second feature information in a similar manner as to how the second feature information of the subject is obtained as described in connection with operation 910. In some embodiments, the processing device 140B may generate sample third feature information of the sample subject based on the sample first feature information and the sample second feature information in a similar manner as to how the third feature information of the subject is obtained as aforementioned. The ground truth severity of illness of the sample subject may be provided or confirmed by a user, and used as a labeled quantized value. In some embodiments, the processing device 140B may perform a selection operation on the sample first feature information, sample second feature information, and/or sample third feature information. More descriptions regarding the selection operation may be found elsewhere in this disclosure. The processing device 140B may designate the selected sample first and second feature information, or the selected sample third feature information as the sample feature information of the fourth training sample. In some embodiments, the fourth training sample may be previously generated, and the processing device 140B may directly obtain the fourth training sample from a storage device where the fourth training sample is stored.

The processing device 140B may further generate the third severity degree determination model by training a fourth preliminary model using the at least one fourth training sample. The fourth preliminary model to be trained may include one or more model parameters, such as the number (or count) of layers, the number (or count) of nodes, a second loss function, or the like, or any combination thereof. Before training, the fourth preliminary model may have one or more initial parameter values of the model parameter(s).

The training of the fourth preliminary model may include one or more iterations to iteratively update the model parameters of the fourth preliminary model based on the fourth training sample(s) until a second termination condition is satisfied in a certain iteration. The second termination condition may be the same as or similar to the first termination condition as described in combination with operation 610, and the descriptions thereof are not repeated here. The second loss function may be used to measure a discrepancy between a severity of illness predicted by the fourth preliminary model in an iteration and the ground truth severity of illness of the sample subject. For example, sample feature information of each fourth training sample may be input into the fourth preliminary model, and the fourth preliminary model may output a predicted severity of illness of the fourth training sample. The second loss function may be used to measure a discrepancy between the predicted severity of illness and the ground truth severity of illness of each fourth training sample.

Exemplary second loss functions may include a focal loss function, a log loss function, a cross-entropy loss, a dice loss function, or the like. If the second termination condition is not satisfied in the current iteration, the processing device 140B may further update the fourth preliminary model to be used in a next iteration according to, for example, a backpropagation algorithm. If the second termination condition is satisfied in the current iteration, the processing device 140B may designate the fourth preliminary model in the current iteration as the third severity degree determination model.

Different subjects with a similar disease may have different clinical signs, for example, some subjects may have severe clinical signs and some subjects may have mild clinical signs. Conventionally, the severity of illness is determined based on clinical information, historical disease information, and disease symptom information of a subject, so as to determine whether the subject needs to be hospitalized. According to some embodiments of the present disclosure, the processing device 140A may generate at least one first segmentation image and at least one second segmentation image based on a target image of the subject, and determine first feature information relating to at least one lesion region and at least one target region of the subject based on the at least one first segmentation image and the at least one second segmentation image. The processing device 140A may further determine the severity of illness of the subject based on first feature information (which may include disease signs obtained from medical image(s)) and second feature information (which may include physical examination indicator(s) and other clinical information) of the subject. Compared with the conventional approach, the systems of the present disclosure may determine a severity of illness based on more information, thereby improving the accuracy and reliability of the severity of illness.

In addition, the first feature information relating to the at least one lesion region and the at least one target region may be determined based on the at least one first segmentation image and the at least one second segmentation image, and hence the subject may be diagnosed based on the first feature information (e.g., some specific parts having lesion regions may be further examined). This may avoid performing examination or diagnosis on the whole subject blindly, and save examination or diagnosis time.

Moreover, based on the determined first feature information and the second feature information, the severity of illness may be determined simply and quickly, which can save time to improve the efficiency of the determination of severity of illness.

In the processes 800 and 900, the processing device 140A may determine a lesion ratio and a HU value distribution of each lesion region as the first feature information. The lesion ratio and the HU value distribution of a lesion region may really reflect the infection, diffusion, or absorption states of the lesion region, and hence, the severity of illness determined based on the first feature information may truly reflect the severity of illness. In this way, the determined severity of illness is more accurate and closer to the real state of the subject.

FIG. 10 is a flowchart illustrating an exemplary process for determining at least one target case according to some embodiments of the present disclosure. In some embodiments, one or more operations of the process 1000 may be performed to achieve at least part of operation 540 as described in connection with FIG. 5.

In 1010, the processing device 140A (e.g., the generation module 408) may obtain a plurality of reference cases.

As used herein, a reference case may be a case of a reference subject. A reference subject may have a similar disease to or a same disease as the subject. For example, if the subject is a patient with lung disease, the reference subject may be a patient with the same lung disease and the reference case may be a lung disease case of the reference subject. In some embodiments, each of the plurality of reference cases may include reference feature information relating to at least one lesion region and/or at least one target region of a reference subject. In some embodiments, the reference feature information may include reference first feature information and reference second feature information. The reference first feature information and the reference second feature information may be similar to the first feature information and the second feature information, respectively. More descriptions regarding the first feature information and the second feature information may be found elsewhere in the present disclosure. See, e.g., FIG. 5, FIG. 8, and FIG. 9.

In some embodiments, the plurality of reference cases may be stored in a database. The database may include multiple disease cases and reference feature information of each disease case. In some embodiments, the disease cases in the database may be collected manually. In some embodiments, the disease cases stored in the database may be confirmed in advance by experts to ensure that the disease cases in the database have a guiding effect. In some embodiments, the reference feature information of a disease case in the database may be determined or obtained in a similar manner as to how the first feature information and the second feature information of the subject are determined or obtained as described elsewhere in this disclosure. In some embodiments, the disease cases in the database may relate to a same disease as or a similar disease to the subject to be analyzed. For example, if the subject has a pneumonia, a database including a plurality of lung disease cases may be used.

In some embodiments, a disease case in the database may include a medical image, a historical disease record, or the like, or any combination thereof. The historical disease record refers to a continuous record for a state of a disease, a diagnosis process, and/or a treatment process of a patient (e.g., a reference subject). In some embodiments, the historical disease record may include a type of a disease, a change of the state of the disease, an examination result, a physicians' ward round record, a consultation opinion, a discussion opinion provided by doctors, a diagnosis and treatment measure and its effect, the change of a doctor's advice and a reason of the change, or the like, or any combination thereof.

In some embodiments, the database may be a local database or an online database. The online database may be connected to a server (e.g., the processing device 140A) through a network, so that hospitals and/or disease research centers may obtain, retrieve, and update data in the online database in real-time or intermittently (e.g., periodically). In some embodiments, new disease cases may be updated into the database to continuously update the database. In some embodiments, the new disease cases may be updated into an off-line or online temporary knowledge database, and then updated into the database after being confirmed by multiple experts to ensure the authority and guidance of the disease cases stored in the database.

In 1020, the processing device 140A (e.g., the generation module 408) may select, from the plurality of reference cases, at least one target case based on the reference feature information of the plurality of reference cases and the first feature information.

The reference subject of each of the at least one target case may have a similar disease to the subject. The count of the at least one target case may be any integer greater than or equal to 1.

In some embodiments, a target case may be a reference case that meets a preset condition. The preset condition may be set manually by a user (e.g., a doctor), or according to a default setting of the imaging system 100, or determined by the processing device 140A according to an actual need. For example, the preset condition may be that a similarity between reference feature information of the reference case and the feature information of the subject exceeds a preset threshold.

In some embodiments, the processing device 140A may determine the at least one target case from the plurality of reference cases based on a similarity between the reference feature information of each of the plurality of reference cases and the first feature information. For example, if a similarity between the reference feature information of a reference case and the first feature information exceeds a threshold, such as 90%, 95%, 99%, etc., the processing device 140A may determine the reference case as a target case. As another example, the processing device 140A may determine the reference case with the largest similarity as the target case.

In some embodiments, the first feature information and the reference feature information may relate to one or more features. For a reference case, the processing device 140A may determine one or more similarities corresponding to the feature(s). For example, the processing device 140A may determine a first similarity relating to a morphological feature (e.g., a lesion ratio) between the reference subject and the subject, and a second similarity relating to a density feature (e.g., a HU value distribution) between the reference subject and the subject based on the reference feature information and the first feature information. If the first and second similarities are both greater than their corresponding preset thresholds, the processing device 140A may determine the reference case as a target case. If at least one of the first and second similarities is less than their corresponding preset thresholds, the processing device 140A may obtain another reference case from a database for storing reference cases, and repeat the above process until a preset number of target cases are obtained or the all reference cases in the database are analyzed. In some embodiments, the processing device 140A may determine a similarity between each reference case in the database and the subject. The processing device 140A may determine at least one reference case with the top N (N being any positive integer) similarity as the at least one target case.

In some embodiments, the similarity between the reference feature information of the reference case and the first feature information may be determined based on feature vectors representing the reference feature information and the first feature information. Specifically, the reference feature information of each of the plurality of reference cases may be represented as a reference feature vector. For example, the processing device 140A may determine a first feature vector representing the first feature information of the subject based on the first feature information. As another example, the processing device 140A may determine third feature information of the subject based on the first feature information and second feature information (e.g., clinical information) of the subject, and determine the first feature vector representing the third feature information of the subject based on the third feature information. In some embodiments, the processing device 140A may determine the reference feature vector and the first feature vector by processing the reference feature information and the first feature information (or the third feature information) using an encoding model, respectively. Exemplary encoding models may include a Bidirectional Encoder Representations from Transformer (BERT) model, a Word Embedding model, a Long Short-Term Memory (LSTM) model, or the like.

Further, the processing device 140A may determine the at least one target case based on the plurality of reference feature vectors and the first feature vector. For example, the processing device 140A may determine the similarity between the reference feature information of a reference case and the first feature information by determining a similarity between the reference feature vector of the reference case and the first feature vector. In some embodiments, the similarity between two vectors may be determined according to a distance between the two vectors. The distance between two vectors may include a cosine distance, a Euclidean distance, a Manhattan distance, a Mahalanobis distance, a Minkowski distance, etc. The distance is negatively correlated to the similarity, that is, the greater the distance, the smaller the similarity. In some embodiments, the processing device 140A may determine the at least one target case based on the plurality of reference feature vectors and the feature vector according to a vector index algorithm. Exemplary vector index algorithms may include a k-dimensional tree (KD-tree) algorithm, a locality-sensitive hashing (LSH) algorithm, an approximate nearest neighbor search (ANNS) algorithm, or threshold like, or any combination thereof.

According to some embodiments of the present disclosure, the processing device 140A may determine the at least one target case from the plurality of reference cases based on the reference feature information of each of the plurality of reference cases and the first feature information. Compared with a conventional approach of manually determining target case(s) by a user, the systems and methods of the present disclosure may be more reliable and robust, insusceptible to human error or subjectivity, and/or fully automated, which may improve the efficiency and the accuracy of the determination of the target case(s). In addition, the processing device 140A may determine the first feature information based on information relating to multiple levels of target regions and lesion regions of the multiple levels of target regions. For example, first feature information relating to the lung lobes and lung segments may be determined. In this way, the at least one target case determined based on the first feature information and the reference feature information of reference cases may be more accurate.

FIGS. 11A and 11B are schematic diagrams illustrating exemplary training processes of a feature extraction model and a diagnosis result generation model according to some embodiments of the present disclosure.

The feature extraction model may be a trained model (e.g., a machine learning model) used for extracting first feature information relating to at least one lesion region and/or at least one target region of a subject. Merely by way of example, at least one first segmentation image and at least one second segmentation image of the at least one target region of the subject may be input into the feature extraction model, and the feature extraction model may output first feature information relating to the at least one lesion region and/or the at least one target region.

The diagnosis result generation model a trained model (e.g., a machine learning model) used for generating a diagnosis result with respect to a subject. Merely by way of example, first feature information and/or second feature information of the subject may be input into the diagnosis result generation model, and the diagnosis result generation model may output the diagnosis result with respect to the subject.

In some embodiments, the diagnosis result generation model may be trained based on a plurality of training samples including sample first feature information. Alternatively, the feature extraction model and the diagnosis result generation model may be jointly trained base on a plurality of training samples including sample first segmentation images and second segmentation images.

As shown in FIG. 11A, the processing device 140B may generate a diagnosis result generation model by training a preliminary diagnosis result generation model based on a plurality of fifth training samples. Each fifth training sample may include sample first feature information of a sample subject and a ground truth diagnosis result of the sample subject. Specifically, model parameters of the preliminary diagnosis result generation model may be iteratively updated based on the fifth training sample(s) until a third loss function of the preliminary diagnosis result generation model meets a preset condition, for example, the third loss function converges, a value of the third loss function is less than a preset value, etc. When the third loss function meets the preset condition in the current iteration, the training of the preliminary diagnosis result generation model may be completed and the processing device 140B may designate the preliminary diagnosis result generation model in the current iteration as the diagnosis result generation model.

In some embodiments, the feature extraction model and the diagnosis result generation model may be jointly trained using a machine learning algorithm. Merely by way of example, as shown in FIG. 11B, the processing device 140B may generate the feature extraction model and the diagnosis result generation model by training a preliminary feature extraction model and a preliminary diagnosis result generation model based on a plurality of sixth training samples. Each of the plurality of sixth training samples may include at least one sample first segmentation image and at least one sample second segmentation image of a sample subject, and a ground truth diagnosis result of the sample subject.

Specifically, the at least one sample first segmentation image and the at least one sample second segmentation image in each sixth training sample may be inputted into the preliminary feature extraction model. The preliminary feature extraction model may generate an output, which may be inputted into the preliminary diagnosis result generation model. The preliminary diagnosis result generation model may output a predicted diagnosis result. The training of the preliminary feature extraction model and the preliminary diagnosis result generation may include one or more iterations to iteratively update the model parameters of the preliminary feature extraction model and the preliminary diagnosis result generation based on the sixth training sample(s) until a third termination condition is satisfied in a certain iteration. Exemplary third termination conditions may be that the value of a fourth loss function obtained in the certain iteration is less than a threshold value, that a certain count of iterations has been performed, that the fourth loss function converges such that the differences of the values of the fourth loss function obtained in a previous iteration and the current iteration within a threshold value, etc. The fourth loss function may be used to measure a discrepancy between a predicted diagnosis result output by the preliminary diagnosis result generation mode in an iteration and the ground truth diagnosis result. If the third termination condition is satisfied in the current iteration, the processing device 140B may designate the preliminary feature extraction model and the preliminary diagnosis result generation model in the current iteration as the feature extraction model and diagnosis result generation model, respectively.

FIG. 13 illustrates an exemplary first segmentation image 1300 according to some embodiments of the present disclosure. As shown in FIG. 13, a region B corresponds to the right lung of a patient, and a region C corresponds to the left lung of the patient. The first segmentation image 1300 indicates the regions corresponding to the left lung and the right lung identified from a lung image of the patient.

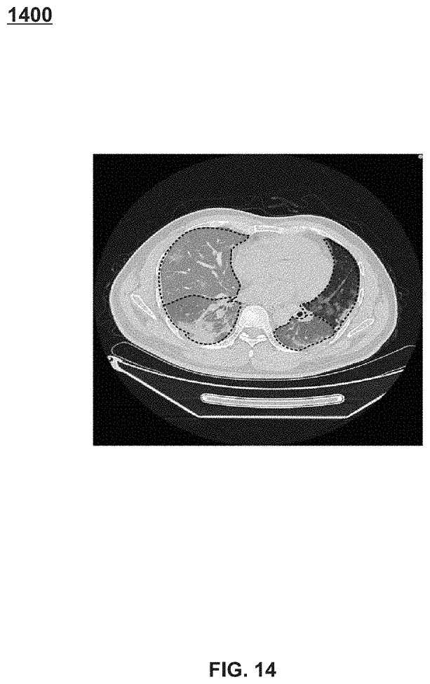

FIG. 14 illustrates an exemplary first segmentation image 1400 according to some embodiments of the present disclosure. As shown in FIG. 14, regions enclosed by the dotted lines on the right represent the lung lobes of the left lung, and regions enclosed by dotted lines on the left represent the lung lobes of the right lung. The first segmentation image 1400 indicates the regions corresponding to the lung lobes of the left lung and the right lung identified from a lung image of the patient.

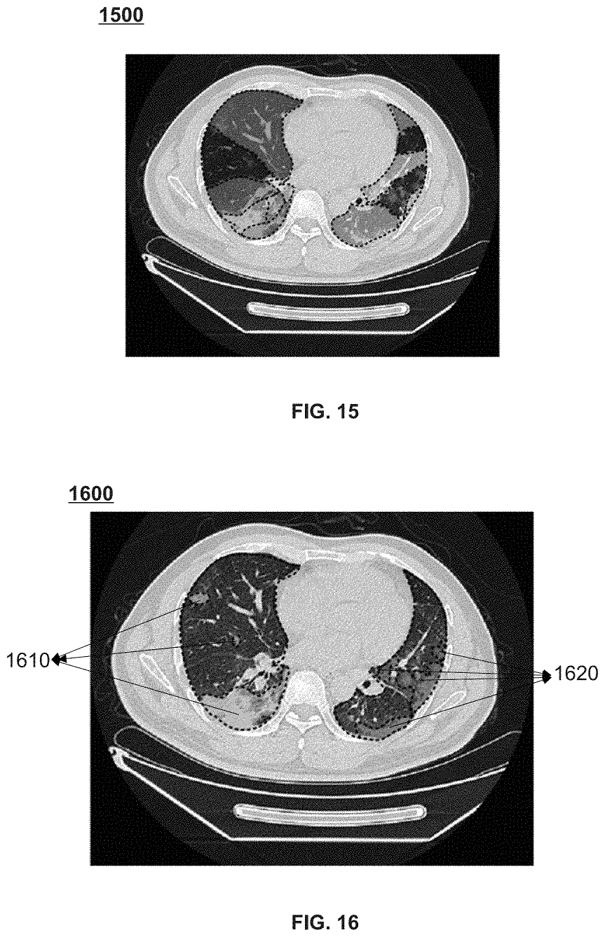

FIG. 15 illustrates an exemplary first segmentation image 1500 according to some embodiments of the present disclosure. As shown in FIG. 15, regions enclosed by the dotted lines on the right represent lung segments of the left lung, and regions enclosed by the dotted lines on the left represent lung segments of the right lung.

FIG. 16 illustrates an exemplary second segmentation image 1600 according to some embodiments of the present disclosure. As shown in FIG. 16, regions 1610 in light grey represent a lesion region of the right lung and regions 1620 in light grey represent a lesion region of the left lung.

It should be noted that the examples shown in FIGS. 13-16 are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, in a first segmentation image, different regions corresponding to different target regions of the patient may be represented by, for example, different colors, different textures, and/or different markers.

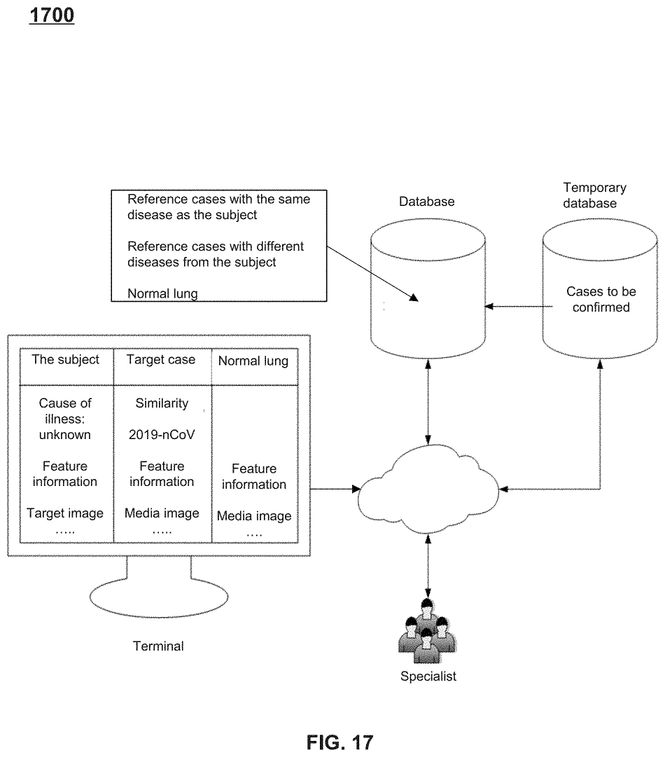

FIG. 17 illustrates an exemplary display result of a target case according to some embodiments of the present disclosure.

As shown in FIG. 17, information relating to the target case may be displayed on a terminal. In some embodiments, information relating to the target case may include a similarity with first feature information of a subject, a cause of the disease of the target case, a state of the disease, a pathology of the disease, a course of the disease, a patient age, the type of basic diseases of the target case, or the like, or any combination thereof. In some embodiments, the target case may need to satisfy a preset condition, which may be set manually by a user, or according to a default setting of the imaging system 100, or determined by the processing device 140A according to an actual need. For example, a user may set a preset condition that the similarity of the target case with the first feature information of the subject is greater than 99%, the disease of the target case is viral pneumonia, and the patient age is over 60 years old according to his/her interest.

In some embodiments, the processing device 140A may select at least one target case and display the at least one target case according to the disease cause, which is helpful to determine the cause of the disease of the subject. Especially for a pneumonia of unknown cause, the processing device 140A may detect the pneumonia in-time and prompt that the cause of the pneumonia is unknown. The state of the pneumonia may include a mild state, a moderate state, a severe state, etc. According to the pathology, the pneumonia may be classified as a lobar pneumonia, a bronchial pneumonia, an interstitial pneumonia, etc. According to the course, the pneumonia may be classified as an acute pneumonia, a persistent pneumonia, a chronic pneumonia, etc. The types of basic diseases may include diseases related to lung diseases, including but not limited to a hypertension, a diabetes, a cardiovascular disease, a chronic obstructive pulmonary disease (COPD), a cancer, a chronic kidney disease, and a hepatitis B, an immunodeficiency disease, or the like, or any combination thereof.

In some embodiments, the processing device 140A may quickly display at least one of the at least one target case that the user is interested in via a terminal according to a preset rule. In some embodiments, a user or the processing device 140A may perform a sort operation or a filtering operation on the at least one target case to quickly determine one or more target cases matching the subject. The features of the determined target case(s), such as the reference feature information, the cause of the disease, the state of the disease, the pathology of the disease, the course of the disease, the patient age, the types of basic diseases, etc., may be similar to the subject. The determination of the target case(s) may be useful for subsequent diagnosis and treatment, or medical research.

In some embodiments, reference cases with the same disease as the subject and reference feature information of the reference cases may be stored in the database. For example, if the disease of the subject is a pneumonia, only reference cases with the pneumonia and reference feature information of the reference cases may be stored in the database. Additionally or alternatively, reference cases with different diseases from the subject and reference feature information of the reference cases may be stored in the database. For example, if the disease of the subject is a pneumonia, reference cases with other diseases (e.g., a pulmonary fibrosis, an emphysema, a lung cancer, etc.) and reference feature information of the reference cases may be stored in the database.

In some embodiments, the reference subject of a target case may have a same disease as the subject or a different disease from the subject. For example, a reference case with a different disease from the subject but similar feature information to the subject may be determine as a target case. Since different types of diseases (e.g., lung diseases) may have similar image signs, a reference case with a different disease from the subject but similar feature information to the subject may provide more reference information for a user to perform subsequent disease diagnosis process.

In some embodiments, the processing device 140A may display information relating to the subject and the information relating to a target case simultaneously to compare the subject and the target case. For example, information of the subject (e.g., a medical image, feature information, a similarity with respect to the target case, the cause of the disease, the state of the disease, the pathology of the disease, the course of the disease, the age of the subject, types of basic diseases) and corresponding information of the target case may be displayed in parallel. A user may distinguish differences between the feature information of the target case and the subject easily. For example, in teaching, users may distinguish differences between the feature information and differences between medical images of the target case and the subject, thereby deepening the learning impression and enhancing the teaching effect.

In some embodiments, the database may include a lung image of a normal subject and feature information of the normal subject. The processing device 140A may display information relating to the subject, information relating to a target case, and information relating to the normal subject simultaneously. A user may distinguish differences between feature information and differences between medical images of the normal subject and the subject. For example, in teaching, users may distinguish differences between the feature information and differences between medical images of the normal subject and the subject, thereby deepening the learning impression and enhancing the teaching effect.

There are many types of lung diseases. For example, a pneumonia may be classified as a bacterial pneumonia, a viral pneumonia, a pneumonia caused by atypical pathogens, a pneumonia with unknown cause, etc., according to the cause of the pneumonia. As another example, the pneumonia may be classified as a lobar pneumonia, a lobular pneumonia, an interstitial pneumonia, etc., according to the anatomy. Moreover, different stages of a pneumonia may also show different symptoms of the disease.

Taking the COVID-19 as an example, early COVID-19 is represented as multiple patchy ground glass-like density lesions scattered in both lungs, mainly around the subpleural lung. Critical COVID-19 is represented as multiple patchy mixed density lesions distributed in both lung segments and lobes, involving the center and periphery of the lungs, and the composition of the ground glass of the lesions is relatively reduced. A disease diagnosis result determined by traditional approaches is not accurate for only using the feature information of the lesion region.

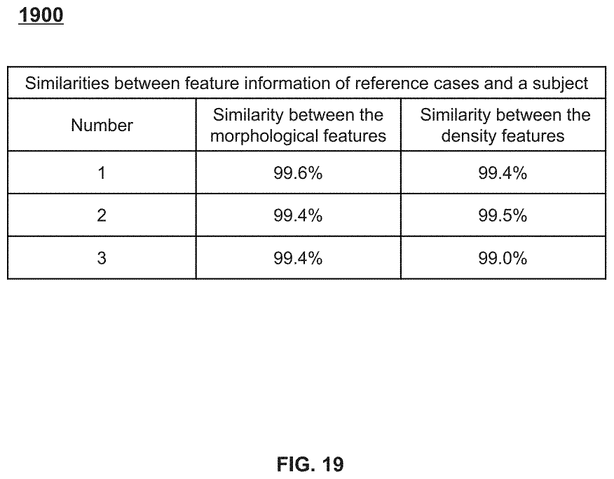

FIG. 19 illustrates exemplary similarities between feature information of reference cases and a subject according to some embodiments of the present disclosure. As shown in FIG. 19, for each reference case, the similarities include a first similarity between morphological features of the subject and the reference subject of the reference case, and a second similarity between density features of the subject and the reference subject. For example, if the first similarity and the second similarity of a reference case are both greater than 99%, the reference case may be selected as a target case. In such cases, the reference cases 1, 2, and 3 in FIG. 19 may be determined as target cases. As another example, if the first similarity of a reference case is greater than 99.5% and the second similarity of the reference case is greater than 99%, the reference case may be selected as a target case. In such cases, the reference case 1 in FIG. 19 may be determined as a target case.

It will be apparent to those skilled in the art that various changes and modifications can be made in the present disclosure without departing from the spirit and scope of the disclosure. In this manner, the present disclosure may be intended to include such modifications and variations if the modifications and variations of the present disclosure are within the scope of the appended claims and the equivalents thereof.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or feature described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or features may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an subject oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Per, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local region network (LAN) or a wide region network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate a certain variation (e.g., ±1%, ±5%, ±10%, or ±20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value.”

What is claimed is:

1. A system, comprising:
at least one storage device including a set of instructions; and
at least one processor configured to communicate with the at least one storage device, wherein when executing the set of instructions, the at least one processor is configured to direct the system to perform operations including:
obtaining a target image of a subject including at least one target region;
generating, based on the target image, at least one first segmentation image and at least one second segmentation image, each of the at least one first segmentation image indicating one of the at least one target region of the subject, each of the at least one second segmentation image indicating a lesion region of one of the at least one target region;
determining, based on the at least one first segmentation image and the at least one second segmentation image, first feature information relating to the at least one lesion region and the at least one target region; and
generating a diagnosis result with respect to the subject based on the first feature information.

2. The system of claim 1, wherein the target image is a medical image of the lungs of the subject, and the at least one target region includes at least one of the left lung, the right lung, a lung lobe, or a lung segment of the subject.

3. The system of claim 1, wherein the diagnosis result with respect to the subject includes a severity of illness of the subject or at least one target case, each of the at least one target case relating to a reference subject having a similar disease to the subject.

4. The system of claim 1, wherein the generating, based on the target image, at least one first segmentation image and at least one second segmentation image comprises:
generating the at least one first segmentation image by processing the target image using a first segmentation model for segmenting the at least one target region; and
generating the at least one second segmentation image by processing the at least one first segmentation image and the target image using a second segmentation model for segmenting the at least one lesion region.

5. The system of claim 1, wherein the generating, based on the target image, at least one first segmentation image and at least one second segmentation image comprises:
generating the at least one first segmentation image by processing the target image using a first segmentation model for segmenting the at least one target region; and
generating the at least one second segmentation image by processing the target image using a third segmentation model for segmenting the at least one lesion region.

6. The system of claim 1, wherein the determining, based on the at least one first segmentation image and the at least one second segmentation image, first feature information relating to the at least one lesion region and the at least one target region comprises:
for each of the at least one lesion region,
determining a lesion ratio of the lesion region to the target region corresponding to the lesion region based on the second segmentation image of the lesion region and the first segmentation image of the target region corresponding to the lesion region;
determining a HU value distribution of the lesion region; and
determining, based on the lesion ratio and the HU value distribution of the lesion region, the first feature information.

7. The system of claim 1, wherein the generating a diagnosis result with respect to the subject based on the first feature information comprises:
obtaining second feature information of the subject, wherein the second feature information includes clinical information of the subject; and
generating the diagnosis result with respect to the subject based on the first feature information and the second feature information.

8. The system of claim 7, wherein the generating the diagnosis result with respect to the subject based on the first feature information and the second feature information comprises:
generating third feature information of the subject based on the first feature information and the second feature information; and
determining a severity of illness of the subject by processing the third feature information using a severity degree determination model.

9. The system of claim 8, wherein the severity degree determination model is generated according to a model training process including:
obtaining at least one training sample each of which includes sample feature information of a sample subject and a ground truth severity of illness of the sample subject, wherein the sample feature information of the sample subject includes sample first feature information relating to at least one sample lesion region and at least one sample target region of the sample subject, and sample second feature information of the sample subject; and
generating the severity degree determination model by training a preliminary model using the at least one training sample.

10. The system of claim 1, wherein generating a diagnosis result with respect to the subject based on the first feature information including:
generating the diagnosis result with respect to the subject by processing the first feature information using a diagnosis result generation model.

11. The system of claim 10, the determining, based on the at least one first segmentation image and the at least one second segmentation image, first feature information relating to the at least one lesion region and the at least one target region comprising:
generating the first feature information by processing the at least one first segmentation image and the at least one second segmentation image using a feature extraction model, wherein the feature extraction model and the diagnosis result generation model are jointly trained using a machine learning algorithm.

12. The system of claim 1, wherein generating a diagnosis result with respect to the subject based on the first feature information comprises:
obtaining a plurality of reference cases, wherein each of the plurality of reference cases includes reference feature information relating to at least one lesion region and at least one target region of a reference subject; and
selecting, from the plurality of reference cases, at least one target case based on the reference feature information of the plurality of reference cases and the first feature information, the reference subject of each of the at least one target case having a similar disease to the subject.

13. The system of claim 12, wherein the reference feature information of each of the plurality of reference cases is represented as a reference feature vector, the selecting, from the plurality of reference cases, at least one target case comprises:
determining, based on the first feature information, a feature vector representing the first feature information of the subject; and
determining the at least one target case based on the plurality of reference feature vectors and the feature vector.

14. The system of claim 13, wherein the determining the at least one target case based on the plurality of reference feature vectors and the feature vector comprises:
determining, based on the plurality of reference feature vectors and the feature vector, the at least one target case according to a Vector Indexing algorithm.

15. The system of claim 1, wherein the determining, based on the at least one first segmentation image and the at least one second segmentation image, first feature information relating to the at least one lesion region and the at least one target region comprises:
determining, based on the at least one first segmentation image and the at least one second segmentation image, initial first feature information; and
generating the first feature information by preprocessing the initial first feature information, wherein the preprocessing of the initial first feature information includes at least one of a normalization operation, a filtering operation, or a weighting operation.

16. A method, the method being implemented on a computing device having at least one storage device and at least one processor, the method comprising:
obtaining a target image of a subject including at least one target region;
generating, based on the target image, at least one first segmentation image and at least one second segmentation image, each of the at least one first segmentation image indicating one of the at least one target region of the subject, each of the at least one second segmentation image indicating a lesion region of one of the at least one target region;
determining, based on the at least one first segmentation image and the at least one second segmentation image, first feature information relating to the at least one lesion region and the at least one target region; and
generating a diagnosis result with respect to the subject based on the first feature information.

17. The method of claim 16, wherein the target image is a medical image of the lungs of the subject, and the at least one target region includes at least one of the left lung, the right lung, a lung lobe, or a lung segment of the subject.

18. The method of claim 16, wherein the diagnosis result with respect to the subject includes a severity of illness of the subject or at least one target case, each of the at least one target case relating to a reference subject having a similar disease to the subject.

19. The method of claim 16, wherein the determining, based on the at least one first segmentation image and the at least one second segmentation image, first feature information relating to the at least one lesion region and the at least one target region comprises:
for each of the at least one lesion region,
determining a lesion ratio of the lesion region to the target region corresponding to the lesion region based on the second segmentation image of the lesion region and the first segmentation image of the target region corresponding to the lesion region;
determining a HU value distribution of the lesion region; and
determining, based on the lesion ratio and the HU value distribution of the lesion region, the first feature information.

20. A non-transitory computer readable medium, comprising a set of instructions, wherein when executed by at least one processor of a computing device, the set of instructions causes the computing device to perform a method, the method comprising:
obtaining a target image of a subject including at least one target region;
generating, based on the target image, at least one first segmentation image and at least one second segmentation image, each of the at least one first segmentation image indicating one of the at least one target region of the subject, each of the at least one second segmentation image indicating a lesion region of one of the at least one target region;
determining, based on the at least one first segmentation image and the at least one second segmentation image, first feature information relating to the at least one lesion region and the at least one target region; and
generating a diagnosis result with respect to the subject based on the first feature information.
